(19) United States
(12) Patent Application Publication
(10) Pub. No.: US 2014/0287528 A1
(43) Pub. Date: Sep. 25, 2014

(54) INCORPORATION OF METHYL LYSINE INTO POLYPEPTIDES

(71) Applicant: MEDICAL RESEARCH COUNCIL, LONDON (GB)

(72) Inventors: Jason Chin, Cambridge (GB); Duy P. Nguyen, Cambridge (GB)

(73) Assignee: MEDICAL RESEARCH COUNCIL, LONDON (GB)

(21) Appl. No.: 14/048,684

(22) Filed: Oct. 8, 2013

Related U.S. Application Data
(63) Continuation of application No. 13/499,450, filed on Jun. 12, 2012, now abandoned.

(30) Foreign Application Priority Data
Oct. 1, 2009 (GB) 0917240.4

(51) Int. Cl. C07K 14/47; G01N 33/566

(52) U.S. Cl. CPC C07K 14/47; G01N 33/566
USPC 436/501; 435/69.1

(57) ABSTRACT

The invention relates to A method of making a polypeptide comprising at least one N^{ε}-methyl-lysine at a specific site in said polypeptide, said method comprising (a) genetically directing the incorporation of R—N^{ε}-methyl-lysine into said polypeptide, wherein R comprises an auxiliary group; and (b) catalysing the removal of R from the polypeptide of (a). In particular the invention relates to such a method wherein genetically directing the incorporation of R—Nε-methyl-lysine into said polypeptide comprises arranging for the translation of a RNA encoding said polypeptide, wherein said RNA comprises an amber codon, and wherein said translation is carried out in the presence of an amber tRNA charged with R—N^{ε}-methyl-lysine.

The present application is a continuation of U.S. patent application Ser. No. 13/499,450, which was filed Jun. 12, 2012, which was filed pursuant to 35 U.S.C. 371 as a U.S. National Phase application of International Patent Application No. PCT/GB2010/001847, which was filed Oct. 1, 2010, claiming the benefit of priority to British Patent Application No. 0917240.4, which was filed on Oct. 1, 2009. The entire text of the aforementioned applications is incorporated herein by reference in its entirety.

### FIELD OF THE INVENTION

The invention relates to genetically encoding N^{e}-methyl-L-lysine in recombinant polypeptides.

### BACKGROUND TO THE INVENTION

The N^{ε}-methylation status of specific lysine residues on histone proteins in chromatin controls heterochromatin formation, X-chromosome inactivation, genome imprinting, DNA repair, regulates transcription and may define epigenetic status¹⁻³. The reversible post-translational methylation of lysine residues in histones is mediated by methylases and demethylases and lysine residues are found in mono-, di- and tri-methylated states. The state and site of modification correlates with functional outcome in ways that are beginning to be deciphered⁴.

A molecular understanding of the organismal phenomena orchestrated by lysine N^{ε}-methylation is impeded by the challenge of producing site-specifically and quantitatively methylated histones. Researchers have used methyltransferases to methylate histones⁵, but in many cases this is unsatisfactory because it is difficult to control the site, extent or degree of methylation using these enzymes in vitro. And in many cases the specific methyltransferase is simply unknown. Native chemical ligation has been used to construct histones with modified N-terminal tails⁶⁻⁸, and this approach has been extended, via multiple ligations, to address ubiquitylation outside the tail of a histone⁹. These experiments are often challenging and require synthesis of large quantities of peptide thioesters. Thioether analogues of N^{ε}-methyl-L-lysine in which the γ-methylene unit of lysine is replaced with a sulfur atom can be installed in proteins^{10, 11}. While these analogues are simple to employ, they are longer than the native amino acids by 0.3 Å¹², decrease the pKₐ of the ammonium protons by 1.1 unit¹³, and have more degrees of freedom—which may lead to altered specificity or affinity in binding interactions¹². Moreover, one method of creating the linkage may also lead to racemization at the alpha carbon of the amino acid¹⁰. Taken together these differences may lead to unpredictable effects on the properties of the analogs. Since these analogs are created for the purpose of discovering unknown properties of the natural system, or explaining known phenomena in molecular detail, differences between the analogs and the natural modification are problematic.

To understand the native system one would ideally install the natural modification via a scalable method that quantitatively introduces the modification at any defined site. We recently demonstrated that another important post-translationally modified amino acid—N^{ε}-acetyl-L-lysine—can be quantitatively and site-specifically genetically encoded in recombinant proteins in response to the amber codon using an evolved pyrrolysyl-tRNA synthetase/tRNA_{CUA} pair that is orthogonal in E. coli¹⁴. This approach is facilitating a molecular understanding of the role of lysine acetylation¹⁵. In principle, it is possible to use a similar approach to evolve an orthogonal tRNA-synthetase/tRNA_{CUA} pair that specifically recognizes methyl-L-lysine (3) and directs its incorporation into recombinant proteins.

### SUMMARY OF THE INVENTION

Creating a synthetase that will use methyl-L-lysine, but discriminate against L-lysine (4, a smaller amino acid which cannot be sterically excluded from the active site, differs by only a single methyl group and is abundant in the cell) by a factor of 10³ to 10⁴ as required for translation is thermodynamically challenging in the absence of an amino acid editing site.^{16, 17}. Indeed the pyrrolysyl-tRNA synthetase does not accept methyl-lysine as a substrate¹⁸ and our efforts to evolve a pyrrolysyl-tRNA synthetase/tRNA_{CUA} pair for the direct genetic encoding of methyl-L-lysine, essentially as previously described, did not yield specific enzymes.

The inventors then had the insight to encode N^{ε}-methyl-L-lysine (3) indirectly by providing the synthetase enzyme with a substrate that was significantly different from both N^{ε}-methyl-L-lysine and L-lysine and then to subsequently effect the facile, quantitative and specific post-translational conversion of this precursor to N^{ε}-methyl-L-lysine on the synthesized protein.

The invention is based on these striking findings.

Thus in one aspect the invention provides a method of making a polypeptide comprising at least one N^{ε}-methyl-lysine at a specific site in said polypeptide, said method comprising

(a) genetically directing the incorporation of R—N^{ε}-methyl-lysine into said polypeptide, wherein R comprises an auxiliary group; and
(b) catalysing the removal of R from the polypeptide of (a).

Suitably genetically directing the incorporation of R—N^{ε}-methyl-lysine into said polypeptide comprises arranging for the translation of a RNA encoding said polypeptide, wherein said RNA comprises an amber codon, and wherein said translation is carried out in the presence of an amber tRNA charged with R—N^{ε}-methyl-lysine.

Suitably the tRNA charged with R—N^{ε}-methyl-lysine is supplied by providing a combination of tRNA capable of being charged with R—N^{ε}-methyl-lysine, a tRNA synthetase capable of charging said tRNA with R—N^{ε}-methyl-lysine, and R—N^{ε}-methyl-lysine.

Suitably the tRNA synthetase capable of charging said tRNA with R—N^{ε}-methyl-lysine comprises Methanosarcina barkeri pyrrolysyl-tRNA synthetase (MbPylRS).

Suitably the tRNA capable of being charged with R—N^{ε}-methyl-lysine comprises Methanosarcina barkeri tRNA_{CUA}. Suitably said tRNA comprises MbtRNA_{CUA} (i.e. suitably said tRNA comprises the publicly available wild type Methanosarcina barkeri tRNA_{CUA} sequence as encoded by the MbPylT gene).

Suitably R comprises tert-butyl-oxycarbonyl.

Suitably removal of R from the polypeptide comprises treatment of the polypeptide with 2% trifluoroacetic acid (TFA) for 4 hours at 37° C.

Suitably the polypeptide comprises a histone.

Suitably the histone is core histone H3.

Suitably the histone is methylated specifically at residue 9.

In another aspect, the invention relates to use of a histone polypeptide produced as described above in monitoring DNA breathing.

In another aspect, the invention relates to a method to determine the effect of a modulator of DNA breathing which comprises the following steps:
i) providing two samples of histone polypeptide produced as described above,
ii) introducing the modulator to one of said samples,
iii) measuring the ability of the histone to bind to heterochromatin protein 1;
   wherein if the histone polypeptide sample of (ii) binds to heterochromatin protein 1 less than the second histone polypeptide sample, then it is determined that the modulator has a dampening effect on DNA breathing.

### DETAILED DESCRIPTION OF THE INVENTION

Lysine methylation is an important post-translational modification of histone proteins that defines epigenetic status, controls heterochromatin formation, X-chromosome inactivation, genome imprinting, DNA repair and transcriptional regulation. Despite considerable efforts by chemical biologists to synthesize modified histones for use in deciphering the molecular role of methylation in these phenomena, no general methods exist in the art to synthesize proteins bearing quantitative site-specific methylation. Here we demonstrate a general method for the quantitative installation of N^{ε}-methyl-L-lysine at defined positions in recombinant histones and demonstrate the use of this method for investigating the methylation dependent binding of HP1 to full length histone H3 mono-methylated on K9 (H3K9me1). This strategy will find wide application in defining the molecular mechanisms by which histone methylation orchestrates cellular phenomena.

### DEFINITIONS

The term ‘comprises’ (comprise, comprising) should be understood to have its normal meaning in the art, i.e. that the stated feature or group of features is included, but that the term does not exclude any other stated feature or group of features from also being present.

N^{ε}-methyl-lysine suitably refers to N^{ε}-methyl-L-lysine.

Suitably the methods of the invention are applied to the site specific installation of N^{ε}-methyl-lysine in a polypeptide. Suitably this is accomplished by genetically encoding the incorporation.

The methods may be applied to any polypeptide of interest. Although many of the examples presented herein are in connection with histone proteins, the skilled reader will appreciate that the methods may be usefully applied to any polypeptide of interest. Histones are one example of a biologically important group of proteins, which in particular have biologically relevant methylation and therefore the invention finds particular application in production of polypeptides for which methylation is known or suspected of having a biologically relevant effect.

Suitably said tRNA comprises MbtRNA_{CUA} (i.e. suitably said tRNA comprises the publicly available wild type Methanosarcina barkeri tRNACUA sequence as encoded by the MbPylT gene).

### Auxiliary Group

The auxiliary group is the removable chemical moiety which forms part of the N^{ε}-methyl-lysine precursor molecule. In other words, the auxiliary group is the R group in the R—N^{ε}-methyl-lysine.

Using N^{ε}-tert-butyl-oxycarbonyl-N^{ε}-methyl-L-lysine as an illustrative example of a N^{ε}-methyl-lysine precursor, the auxiliary group or R group (sometime just referred to as “R”) is the tert-butyl-oxycarbonyl moiety.

The key is that removal of the R group leaves N^{ε}-methyl-lysine. Thus the R group may be any suitable chemical moiety which can be attached to N^{ε}-methyl-lysine. The two most important properties of the R group are

1) that it permits the tRNA synthetase to distinguish between R—N^{ε}-methyl-lysine and naturally occurring amino acids such as L-lysine. Thus the R group needs to be of sufficient size and/or sufficiently different in shape or structure to permit reasonable levels of discrimination over L-lysine.
2) that it is removable to leave N^{ε}-methyl-lysine in the polypeptide of interest.

Removal of the R group may be by any suitable means known in the art. Suitably mild chemical treatment is used to remove the R group whilst not significantly altering the chemical structure of the rest of the polypeptide.

Removal of the R group may be by enzymatic means.

Most suitably removal or the R group is by mild chemical conditions such as comprising treatment of the polypeptide with 2% trifluoroacetic acid (TFA) for 4 hours at 37° C.

Conditions and/or times for removal may easily be optimised by the skilled worker. Suitably the conditions and/or times used are chosen to maximise removal of the R- group whilst minimising any other chemical changes which might be catalysed by the treatment. The effects of the treatment may be easily monitored using (for example) mass spectrometry (MS) techniques as described in the examples section.

Orthogonal tRNA Synthetase-Orthogonal tRNA Pairs

Networks of molecular interactions in organisms have evolved through duplication of a progenitor gene followed by the acquisition of a novel function in the duplicated copy. Described herein are processes that exploit or involve orthogonal molecules: that is, molecules that can process information in parallel with their progenitors without cross-talk between the progenitors and the duplicated molecules. Using these processes, it is now possible to tailor the evolutionary fates of a pair of duplicated molecules from amongst the many natural fates to give a predetermined relationship between the duplicated molecules and the progenitor molecules from which they are derived. This is exemplified herein by the generation of orthogonal tRNA synthetase-orthogonal tRNA pairs that can process information in parallel with wild-type tRNA synthetases and tRNAs but that do not engage in cross-talk between the wild-type and orthogonal molecules. In some embodiments the tRNA and/or synthetase itself may retain its wild type sequence. In those embodiments, suitably said entity retaining its wild type sequence is used in a heterologous setting i.e. in a background or host cell different from its naturally occurring wild type host cell. In this way, the wild type entity may be orthogonal in a functional sense without needing to be structurally altered. Orthogonality and the accepted criteria for same are discussed in more detail below.

The Methanosarcina barkeri PylS gene encodes the MbPylRS tRNA synthetase protein. The Methanosarcina barkeri PylT gene encodes the MbtRNA_{CUA} tRNA.

There are two closely related known aminoacyl-tRNA synthetase sequences, which we designated AcKRS-1 and AcKRS-2 (see WO2009/056803). AcKRS-1 has five mutations (L266V, L270I, Y271F, L274A, C313F) while AcKRS-2 has four mutations (L270I, Y271L, L274A, C313F) with respect to MbPylRS. In addition there are synthetase sequences which may be used in the present invention which are chartacterised by comprising the L266M mutation. An example of such a synthetase sequence is one which comprises L266M, L270I, Y271F, L274A, and C313F mutations; this sequence may be referred to as AcKRS-3. Most suitably the wild type sequences may be used in the methods of the invention.

### Sequence Homology/Identity

Although sequence homology can also be considered in terms of functional similarity (i.e., amino acid residues having similar chemical properties/functions), in the context of the present document it is preferred to express homology in terms of sequence identity.

Sequence comparisons can be conducted by eye or, more usually, with the aid of readily available sequence comparison programs. These publicly and commercially available computer programs can calculate percent homology (such as percent identity) between two or more sequences.

Percent identity may be calculated over contiguous sequences, i.e., one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example in an otherwise identical pair of sequences, one insertion or deletion will cause the following amino acid residues to be put out of alignment, thus potentially resulting in a large reduction in percent homology (percent identity) when a global alignment (an alignment across the whole sequence) is performed. Consequently, most sequence comparison methods are designed to produce optimal alignments that take into consideration possible insertions and deletions without penalising unduly the overall homology (identity) score. This is achieved by inserting “gaps” in the sequence alignment to try to maximise local homology/identity.

These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package, FASTA (Altschul et al., 1990, J. Mol. Biol. 215:403-410) and the GENEWORKS suite of comparison tools.

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62. Once the software has produced an optimal alignment, it is possible to calculate percent homology, preferably percent sequence identity. The software typically does this as part of the sequence comparison and generates a numerical result.

In the context of the present document, a homologous amino acid sequence is taken to include an amino acid sequence which is at least 15, 20, 25, 30, 40, 50, 60, 70, 80 or 90% identical, preferably at least 95 or 98% identical at the amino acid level. Suitably this identity is assessed over at least 50 or 100, preferably 200, 300, or even more amino acids with the relevant polypeptide sequence(s) disclosed herein, most suitably with the full length progenitor (parent) tRNA synthetase sequence. Suitably, homology should be considered with respect to one or more of those regions of the sequence known to be essential for protein function rather than non-essential neighbouring sequences. This is especially important when considering homologous sequences from distantly related organisms.

Most suitably sequence identity should be judged across at least the contiguous region from L266 to C313 of the amino acid sequence of MbPylRS, or the corresponding region in an alternate tRNA synthetase.

The same considerations apply to nucleic acid nucleotide sequences, such as tRNA sequence(s).

### Reference Sequence

When particular amino acid residues are referred to using numeric addresses, the numbering is taken using MbPylRS (Methanosarcina barkeri pyrrolysyl-tRNA synthetase) amino acid sequence as the reference sequence (i.e. as encoded by the publicly available wild type Methanosarcina barkeri PylS gene Accession number Q46E77):

MDKKPLDVLI SATGLWMSRT GTLHKIKHYE VSRSKIYIEM
ACGDHLVVNN SRSCRTARAF RHHKYRKTCK RCRVSDEDIN
NFLTRSTEGK TSVKVKVVSA PKVKKAMPKS VSRAPKPLEN
PVSAKASTDT SRSVPSPAKS TPNSPVPTSA PAPSLTRSQL
DRVEALLSPE DKISLNIAKP FRELESELVT RRKNDFQRLY
TNDREDYLGK LERDITKFFV DRDFLEIKSP ILIPAEYVER
MGINNDTELS KQIFRVDKNL CLRPMLAPTL YNYLRKLDRI
LPDPIKIFEV GPCYRKESDG KEHLEEFTMV NFCQMGSGCT
RENLESLIKE FLDYLEIDFE IVGDSCMVYG DTLDIMHGDL
ELSSAVVGPV PLDREWGIDK PWIGAGFGLE RLLKVMHGFK
NIKRASRSES YYNGISTNL

This is to be used as is well understood in the art to locate the residue of interest. This is not always a strict counting exercise—attention must be paid to the context. For example, if the protein of interest is of a slightly different length, then location of the correct residue in that sequence correseponding to (for example) Y271 may require the sequences to be aligned and the equivalent or corresponding residue picked, rather than simply taking the 271^{st} residue of the sequence of interest. This is well within the ambit of the skilled reader.

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, suitably a randomisation of said site is used, for example as described herein in connection with the evolution and adaptation of tRNA synthetase of the invention. As a default mutation, alanine (A) may be used. Suitably the mutations used at particular site(s) are as set out herein.

A fragment is suitably at least 10 amino acids in length, suitably at least 25 amino acids, suitably at least 50 amino acids, suitably at least 100 amino acids, suitably at least 200 amino acids, suitably at least 250 amino acids, suitably at least 300 amino acids, suitably at least 313 amino acids, or suitably the majority of the tRNA synthetase polypeptide of interest.

### Polypeptides of the Invention

Suitably the polypeptide comprising N^{ε}-methyl lysine is a nucleosome or a nucleosomal polypeptide.

Suitably the polypeptide comprising N^{ε}-methyl lysine is a chromatin or a chromatin associated polypeptide.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

Vectors of the invention may be transformed or transfected into a suitable host cell as described to provide for expression of a protein of the invention. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid. Vectors may be used, for example, to transfect or transform a host cell.

Control sequences operably linked to sequences encoding the protein of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

### Protein Expression and Purification

Host cells comprising polynucleotides of the invention may be used to express proteins of the invention. Host cells may be cultured under suitable conditions which allow expression of the proteins of the invention. Expression of the proteins of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Proteins of the invention can be extracted from host cells by a variety of techniques known in the art, including enzymatic, chemical and/or osmotic lysis and physical disruption.

### Optimisation

Unnatural amino acid incorporation in in vitro translation reactions can be increased by using S30 extracts containing a thermally inactivated mutant of RF-1. Temperature sensitive mutants of RF-1 allow transient increases in global amber suppression in vivo. Increases in tRNA_{CUA} gene copy number and a transition from minimal to rich media may also provide improvement in the yield of proteins incorporating an unnatural amino acid in E. coli.

### Industrial Application

N^{ε}-methylation regulates diverse cellular processes. Lysine methylation is an important post-translational modification of histone proteins that defines epigenetic status, controls heterochromatin formation, X-chromosome inactivation, genome imprinting, DNA repair and transcriptional regulation. Thus, there are clear utilities and industrial applications for the methods and materials disclosed herein, both in the production of saleable products and in facilitation of the study of essential biological processes as noted above.

### Further Applications

Polypeptides of the present invention may possess other post-translational modifications such as acetylation. In this embodiment, inhibition of deacetylase may be advantageous and may be carried out by any suitable method known to those skilled in the art. Suitably inhibition is by gene deletion or disruption of endogenous deacetylase(s). Suitably such disrupted/deleted acetylase is CobB. Suitably inhibition is by inhibition of expression such as inhibition of translation of endogenous deacetylase(s). Suitably inhibition is by addition of exogenous inhibitor such as nicotinamide.

In one aspect the invention relates to the addition of N^{ε}-methyl-lysine to the genetic code of organisms such as Escherichia coli.

The invention finds particular application in synthesis of nucleosomes and/or chromatin bearing N^{ε}-methyl-lysine at defined sites on particular histones. One example of such an application is for determining the effect of defined modifications on nucleosome and chromatin structure and function^{1, 26}.

Since MbPylRS does not recognize the anticodon of MbtRNA_{CUA}¹⁸ it is further possible to combine evolved MbPylRS/MbtRNA pairs with other evolved orthogonal aminoacyl-tRNA synthetase/tRNA_{CUA} pairs, and/or with orthogonal ribosomes with evolved decoding properties²⁷ to direct the efficient incorporation of multiple distinct useful unnatural amino acids in a single protein.

### Further Applications

In one aspect the invention may relate to a method to determine the status of methylation of a histone polypeptide, which comprises the following steps:
i) measuring the ability of the histone to bind to heterochromatin protein land
ii) if the histone binds to heterochromatin protein 1, then determining that it is methylated.
   tRNA Synthetases

The tRNA synthetase of the invention may be varied. Although specific tRNA synthetase sequences may have been used in the examples, the invention is not intended to be confined only to those examples.

In principle any tRNA synthetase which provides the same tRNA charging (aminoacylation) function can be employed in the invention. In this case, it is the ability to charge a tRNA with R—N^{ε}-methyl-lysine which is important.

For example the tRNA synthetase may be from any suitable species such as from archea, for example from Methanosarcina barkeri MS; Methanosarcina barkeri str. Fusaro; Methanosarcina mazei Go1; Methanosarcina acetivorans C2A; Methanosarcina thermophila; or Methanococcoides burtonii. Alternatively the the tRNA synthetase may be from bacteria, for example from Desulfitobacterium hafniense DCB-2; Desulfitobacterium hafniense Y51; Desulfitobacterium hafniense PCP1; Desulfotomaculum acetoxidans DSM 771.

Exemplary sequences from these organisms are the publically available sequences. The following examples are provided as exemplary sequences for pyrrolysine tRNA synthetases:

>M. barkeriMS/1-419/
Methanosarcina barkeri MS
VERSION Q6WRH6.1 GI: 74501411
MDKKPLDVLISATGLWMSRTGTLHKIKHHEVSRSKIYIEMACGDHLVVNNSRSCRTARAFRHHKYRKTCK
RCRVSDEDINNFLTRSTESKNSVKVRVVSAPKVKKAMPKSVSRAPKPLENSVSAKASTNTSRSVPSPAKS
TPNSSVPASAPAPSLTRSQLDRVEALLSPEDKISLNMAKPFRELEPELVTRRKNDFQRLYTNDREDYLGK
LERDITKFFVDRGFLEIKSPILIPAEYVERMGINNDTELSKQIFRVDKNLCLRPMLAPTLYNYLRKLDRI
LPGPIKIFEVGPCYRKESDGKEHLEEFTMVNFCQMGSGCTRENLEALIKEFLDYLEIDFEIVGDSCMVYG
DTLDIMHGDLELSSAVVGPVSLDREWGIDKPWIGAGFGLERLLKVMHGFKNIKRASRSESYYNGISTNL
>M. barkeriF/1-419/
Methanosarcina barkeri str. Fusaro
VERSION YP_304395.1 GI: 73668380
MDKKPLDVLISATGLWMSRTGTLHKIKHYEVSRSKIYIEMACGDHLVVNNSRSCRTARAFRHHKYRKTCK
RCRVSDEDINNFLTRSTEGKTSVKVKVVSAPKVKKAMPKSVSRAPKPLENPVSAKASTDTSRSVPSPAKS
TPNSPVPTSAPAPSLTRSQLDRVEALLSPEDKISLNIAKPFRELESELVTRRKNDFQRLYTNDREDYLGK
LERDITKFFVDRDFLEIKSPILIPAEYVERMGINNDTELSKQIFRVDKNLCLRPMLAPTLYNYLRKLDRI
LPDPIKIFEVGPCYRKESDGKEHLEEFTMVNFCQMGSGCTRENLESLIKEFLDYLEIDFEIVGDSCMVYG
DTLDIMHGDLELSSAVVGPVPLDREWGIDKPWIGAGFGLERLLKVMHGFKNIKRASRSESYYNGISTNL
>M. mazei/1-454
Methanosarcina mazei Go1
VERSION NP_633469.1 GI: 21227547
MDKKPLNTLISATGLWMSRTGTIHKIKHHEVSRSKIYIEMACGDHLVVNNSRSSRTARALRHHKYRKTCK
RCRVSDEDLNKFLTKANEDQTSVKVKVVSAPTRTKKAMPKSVARAPKPLENTEAAQAQPSGSKFSPAIPV
STQESVSVPASVSTSISSISTGATASALVKGNTNPITSMSAPVQASAPALTKSQTDRLEVLLNPKDEISL
NSGKPFRELESELLSRRKKDLQQIYAEERENYLGKLEREITRFFVDRGFLEIKSPILIPLEYIERMGIDN
DTELSKQIFRVDKNFCLRPMLAPNLYNYLRKLDRALPDPIKIFEIGPCYRKESDGKEHLEEFTMLNFCQM
GSGCTRENLESIITDFLNHLGIDFKIVGDSCMVYGDTLDVMHGDLELSSAVVGPIPLDREWGIDKPWIGA
GFGLERLLKVKHDFKNIKRAARSESYYNGISTNL
>M. acetivorans/1-443
Methanosarcina acetivorans C2A
VERSION NP_615128.2 GI: 161484944
MDKKPLDTLISATGLWMSRTGMIHKIKHHEVSRSKIYIEMACGERLVVNNSRSSRTARALRHHKYRKTCR
HCRVSDEDINNFLTKTSEEKTTVKVKVVSAPRVRKAMPKSVARAPKPLEATAQVPLSGSKPAPATPVSAP
AQAPAPSTGSASATSASAQRMANSAAAPAAPVPTSAPALTKGQLDRLEGLLSPKDEISLDSEKPFRELES
ELLSRRKKDLKRIYAEERENYLGKLEREITKFFVDRGFLEIKSPILIPAEYVERMGINSDTELSKQVFRI
DKNFCLRPMLAPNLYNYLRKLDRALPDPIKIFEIGPCYRKESDGKEHLEEFTMLNFCQMGSGCTRENLEA
IITEFLNHLGIDFEIIGDSCMVYGNTLDVMHDDLELSSAVVGPVPLDREWGIDKPWIGAGFGLERLLKVM
HGFKNIKRAARSESYYNGISTNL
>M. thermophila/1-478
Methanosarcina thermophila, VERSION DQ017250.1 GI: 67773308
MDKKPLNTLISATGLWMSRTGKLHKIRHHEVSKRKIYIEMECGERLVVNNSRSCRAARALRHHKYRKICK
HCRVSDEDLNKFLTRTNEDKSNAKVTVVSAPKIRKVMPKSVARTPKPLENTAPVQTLPSESQPAPTTPIS
ASTTAPASTSTTAPAPASTTAPAPASTTAPASASTTISTSAMPASTSAQGTTKFNYISGGFPRPIPVQAS
APALTKSQIDRLQGLLSPKDEISLDSGTPFRKLESELLSRRRKDLKQIYAEEREHYLGKLEREITKFFVD
RGFLEIKSPILIPMEYIERMGIDNDKELSKQIFRVDNNFCLRPMLAPNLYNYLRKLNRALPDPIKIFEIG
PCYRKESDGKEHLEEFTMLNFCQMGSGCTRENLEAIIKDFLDYLGIDFEIVGDSCMVYGDTLDVMHGDLE
LSSAVVGPVPMDRDWGINKPWIGAGFGLERLLKVMHNFKNIKRASRSESYYNGISTNL
>M. burtonii/1-416
Methanococcoides burtonii DSM 6242, VERSION YP_566710.1 GI: 91774018
MEKQLLDVLVELNGVWLSRSGLLHGIRNFEITTKHIHIETDCGARFTVRNSRSSRSARSLRHNKYRKPCK
RCRPADEQIDRFVKKTFKEKRQTVSVFSSPKKHVPKKPKVAVIKSFSISTPSPKEASVSNSIPTPSISVV
KDEVKVPEVKYTPSQIERLKTLMSPDDKIPIQDELPEFKVLEKELIQRRRDDLKKMYEEDREDRLGKLER
DITEFFVDRGFLEIKSPIMIPFEYIERMGIDKDDHLNKQIFRVDESMCLRPMLAPCLYNYLRKLDKVLPD
PIRIFEIGPCYRKESDGSSHLEEFTMVNFCQMGSGCTRENMEALIDEFLEHLGIEYEIEADNCMVYGDTI
DIMHGDLELSSAVVGPIPLDREWGVNKPWMGAGFGLERLLKVRHNYTNIRRASRSELYYNGINTNL
>D. hafniense_DCB-2/1-279
Desulfitobacterium hafniense DCB-2
VERSION YP_002461289.1 GI: 219670854
MSSFWTKVQYQRLKELNASGEQLEMGFSDALSRDRAFQGIEHQLMSQGKRHLEQLRTVKHRPALLELEEG
LAKALHQQGFVQVVTPTIITKSALAKMTIGEDHPLFSQVFWLDGKKCLRPMLAPNLYTLWRELERLWDKP
IRIFEIGTCYRKESQGAQHLNEFTMLNLTELGTPLEERHQRLEDMARWVLEAAGIREFELVTESSVVYGD
TVDVMKGDLELASGAMGPHFLDEKWEIVDPWVGLGFGLERLLMIREGTQHVQSMARSLSYLDGVRLNIN
>D. hafniense_Y51/1-312
Desulfitobacterium hafniense Y51
VERSION YP_521192.1 GI: 89897705
MDRIDHTDSKFVQAGETPVLPATFMFLTRRDPPLSSFWTKVQYQRLKELNASGEQLEMGFSDALSRDRAF
QGIEHQLMSQGKRHLEQLRTVKHRPALLELEEGLAKALHQQGFVQVVTPTIITKSALAKMTIGEDHPLFS
QVFWLDGKKCLRPMLAPNLYTLWRELERLWDKPIRIFEIGTCYRKESQGAQHLNEFTMLNLTELGTPLEE
RHQRLEDMARWVLEAAGIREFELVTESSVVYGDTVDVMKGDLELASGAMGPHFLDEKWEIVDPWVGLGFG
LERLLMIREGTQHVQSMARSLSYLDGVRLNIN
>D. hafniensePCP1/1-288
Desulfitobacterium hafniense
VERSION AY692340.1 GI: 53771772
MFLTRRDPPLSSFWTKVQYQRLKELNASGEQLEMGFSDALSRDRAFQGIEHQLMSQGKRHLEQLRTVKHR
PALLELEEKLAKALHQQGFVQVVTPTIITKSALAKMTIGEDHPLFSQVFWLDGKKCLRPMLAPNLYTLWR
ELERLWDKPIRIFEIGTCYRKESQGAQHLNEFTMLNLTELGTPLEERHQRLEDMARWVLEAAGIREFELV
TESSVVYGDTVDVMKGDLELASGAMGPHFLDEKWEIFDPWVGLGFGLERLLMIREGTQHVQSMARSLSYL
DGVRLNIN
>D. acetoxidans/1-277
Desulfotomaculum acetoxidans DSM 771
VERSION YP_003189614.1 GI: 258513392
MSFLWTVSQQKRLSELNASEEEKNMSFSSTSDREAAYKRVEMRLINESKQRLNKLRHETRPAICALENRL
AAALRGAGFVQVATPVILSKKLLGKMTITDEHALFSQVFWIEENKCLRPMLAPNLYYILKDLLRLWEKPV
RIFEIGSCFRKESQGSNHLNEFTMLNLVEWGLPEEQRQKRISELAKLVMDETGIDEYHLEHAESVVYGET
VDVMHRDIELGSGALGPHFLDGRWGVVGPWVGIGFGLERLLMVEQGGQNVRSMGKSLTYLDGVRLNI

When the particular tRNA charging (aminoacylation) function has been provided by mutating the tRNA synthetase, then it may not be appropriate to simply use another wild-type tRNA sequence, for example one selected from the above. In this scenario, it will be important to preserve the same tRNA charging (aminoacylation) function. This is accomplished by transferring the mutation(s) in the exemplary tRNA synthetase into an alternate tRNA synthetase backbone, such as one selected from the above.

In this way it should be possible to transfer selected mutations to corresponding tRNA synthetase sequences such as corresponding pylS sequences from other organisms beyond exemplary M. barkeri and/or M. mazei sequences.

Target tRNA synthetase proteins/backbones, may be selected by alignment to known tRNA synthetases such as exemplary M. barkeri and/or M. mazei sequences.

This subject is now illustrated by reference to the pylS (pyrrolysine tRNA synthetase) sequences but the principles apply equally to the particular tRNA synthetase of interest.

For example, FIG. 9 provides an alignment of all PylS sequences. These can have a low overall % sequence identity. Thus it is important to study the sequence such as by aligning the sequence to known tRNA synthetases (rather than simply to use a low sequence identity score) to ensure that the sequence being used is indeed a tRNA synthetase.

Thus suitably when sequence identity is being considered, suitably it is considered across the tRNA synthetases as in FIG. 9. Suitably the % identity may be as defined from FIG. 9. FIG. 2 shows a diagram of sequence identities between the tRNA synthetases. Suitably the % identity may be as defined from FIG. 10.

It may be useful to focus on the catalytic region. FIG. 11 aligns just the catalytic regions. The aim of this is to provide a tRNA catalytic region from which a high % identity can be defined to capture/identify backbone scaffolds suitable for accepting mutations transplanted in order to produce the same tRNA charging (aminoacylation) function, for example new or unnatural amino acid recognition.

Thus suitably when sequence identity is being considered, suitably it is considered across the catalytic region as in FIG. 11. Suitably the % identity may be as defined from FIG. 11. FIG. 4 shows a diagram of sequence identities between the catalytic regions. Suitably the % identity may be as defined from FIG. 12.

‘Transferring’ or ‘transplanting’ mutations onto an alternate tRNA synthetase backbone can be accomplished by site directed mutagenesis of a nucleotide sequence encoding the tRNA synthetase backbone. This technique is well known in the art. Essentially the backbone pylS sequence is selected (for example using the active site alignment discussed above) and the selected mutations are transferred to (i.e. made in) the corresponding/homologous positions.

When particular amino acid residues are referred to using numeric addresses, unless otherwise apparent, the numbering is taken using MbPylRS (Methanosarcina barkeri pyrrolysyl-tRNA synthetase) amino acid sequence as the reference sequence (i.e. as encoded by the publicly available wild type Methanosarcina barkeri PylS gene Accession number Q46E77):

MDKKPLDVLI SATGLWMSRT GTLHKIKHYE VSRSKIYIEM
ACGDHLVVNN SRSCRTARAF RHHKYRKTCK RCRVSDEDIN
NFLTRSTEGK TSVKVKVVSA PKVKKAMPKS VSRAPKPLEN
PVSAKASTDT SRSVPSPAKS TPNSPVPTSA PAPSLTRSQL
DRVEALLSPE DKISLNIAKP FRELESELVT RRKNDFQRLY
TNDREDYLGK LERDITKFFV DRDFLEIKSP ILIPAEYVER
MGINNDTELS KQIFRVDKNL CLRPMLAPTL YNYLRKLDRI
LPDPIKIFEV GPCYRKESDG KEHLEEFTMV NFCQMGSGCT
RENLESLIKE FLDYLEIDFE IVGDSCMVYG DTLDIMHGDL
ELSSAVVGPV PLDREWGIDK PWIGAGFGLE RLLKVMHGFK
NIKRASRSES YYNGISTNL

This is to be used as is well understood in the art to locate the residue of interest. This is not always a strict counting exercise—attention must be paid to the context or alignment. For example, if the protein of interest is of a slightly different length, then location of the correct residue in that sequence corresponding to (for example) L266 may require the sequences to be aligned and the equivalent or corresponding residue picked, rather than simply taking the 266th residue of the sequence of interest. This is well within the ambit of the skilled reader.

Notation for mutations used herein is the standard in the art. For example L266M means that the amino acid corresponding to L at position 266 of the wild type sequence is replaced with M.

The transplantation of mutations between alternate tRNA backbones is now illustrated with reference to exemplary M. barkeri and M. mazei sequences, but the same principles apply equally to transplantation onto or from other backbones.

For example Mb AcKRS is an engineered synthetase for the incorporation of AcK Parental protein/backbone: M. barkeri PylS

### Mutations: L266V, L270I, Y271F, L274A, C317F

Mb PCKRS: engineered synthetase for the incorporation of PCK
Parental protein/backbone: M. barkeri PylS

### Mutations: M241F, A267S, Y271C, L274M

Synthetases with the same substrate specificities can be obtained by transplanting these mutations into M. mazei PylS. The sequence homology of the two synthetases can be seen in FIG. 13. Thus the following synthetases may be generated by transplantation of the mutations from the Mb backbone onto the Mm tRNA backbone:

Mm AcKRS introducing mutations L301V, L305I, Y306F, L309A, C348F into M. mazei PylS, and
Mm PCKRS introducing mutations M276F, A302S, Y306C, L309M into M. mazei PylS.

Full length sequences of these exemplary transplanted mutation synthetases are given below.

>Mb_PyIS/1-419
MDKKPLDVLISATGLWMSRTGTLHKIKHHEVSRSKIYIEMACGDHLVVNNSRSCRTARAFRHHKYRKTCKRCR
VSDEDINNFLTRSTESKNSVKVRVVSAPKVKKAMPKSVSRAPKPLENSVSAKASTNTSRSVPSPAKSTPNSSV
PASAPAPSLTRSQLDRVEALLSPEDKISLNMAKPFRELEPELVTRRKNDFQRLYTNDREDYLGKLERDITKFF
VDRGFLEIKSPILIPAEYVERMGINNDTELSKQIFRVDKNLCLRPMLAPTLYNYLRKLDRILPGPIKIFEVGP
CYRKESDGKEHLEEFTMVNFCQMGSGCTRENLEALIKEFLDYLEIDFEIVGDSCMVYGDTLDIMHGDLELSSA
VVGPVSLDREWGIDKPWIGAGFGLERLLKVMHGFKNIKRASRSESYYNGISTNL
>Mb_AcKRS/1-419
MDKKPLDVLISATGLWMSRTGTLHKIKHHEVSRSKIYIEMACGDHLVVNNSRSCRTARAFRHHKYRKTCKRCR
VSGEDINNFLTRSTESKNSVKVRVVSAPKVKKAMPKSVSRAPKPLENSVSAKASTNTSRSVPSPAKSTPNSSV
PASAPAPSLTRSQLDRVEALLSPEDKISLNMAKPFRELEPELVTRRKNDFQRLYTNDREDYLGKLERDITKFF
VDRGFLEIKSPILIPAEYVERMGINNDTELSKQIFRVDKNLCLRPMVAPTIFNYARKLDRILPGPIKIFEVGP
CYRKESDGKEHLEEFTMVNFFQMGSGCTRENLEALIKEFLDYLEIDFEIVGDSCMVYGDTLDIMHGDLELSSA
VVGPVSLDREWGIDKPWIGAGFGLERLLKVMHGFKNIKRASRSESYYNGISTNL
>Mb_PCKRS/1-419
MDKKPLDVLISATGLWMSRTGTLHKIKHHEVSRSKIYIEMACGDHLVVNNSRSCRTARAFRHHKYRKTCKRCR
VSDEDINNFLTRSTESKNSVKVRVVSAPKVKKAMPKSVSRAPKPLENSVSAKASTNTSRSVPSPAKSTPNSSV
PASAPAPSLTRSQLDRVEALLSPEDKISLNMAKPFRELEPELVTRRKNDFQRLYTNDREDYLGKLERDITKFF
VDRGFLEIKSPILIPAEYVERFGINNDTELSKQIFRVDKNLCLRPMLSPTLCNYMRKLDRILPGPIKIFEVGP
CYRKESDGKEHLEEFTMVNFCQMGSGCTRENLEALIKEFLDYLEIDFEIVGDSCMVYGDTLDIMHGDLELSSA
VVGPVSLDREWGIDKPWIGAGFGLERLLKVMHGFKNIKRASRSESYYNGISTNL
>Mm_PyIS/1-454
MDKKPLNTLISATGLWMSRTGTIHKIKHHEVSRSKIYIEMACGDHLVVNNSRSSRTARALRHHKYRKTCKRCR
VSDEDLNKFLTKANEDQTSVKVKVVSAPTRTKKAMPKSVARAPKPLENTEAAQAQPSGSKFSPAIPVSTQESV
SVPASVSTSISSISTGATASALVKGNTNPITSMSAPVQASAPALTKSQTDRLEVLLNPKDEISLNSGKPFREL
ESELLSRRKKDLQQIYAEERENYLGKLEREITRFFVDRGFLEIKSPILIPLEYIERMGIDNDTELSKQIFRVD
KNFCLRPMLAPNLYNYLRKLDRALPDPIKIFEIGPCYRKESDGKEHLEEFTMLNFCQMGSGCTRENLESIITD
FLNHLGIDFKIVGDSCMVYGDTLDVMHGDLELSSAVVGPIPLDREWGIDKPWIGAGFGLERLLKVKHDFKNIK
RAARSESYYNGISTNL
>Mm_AcKRS/1-454
MDKKPLNTLISATGLWMSRTGTIHKIKHHEVSRSKIYIEMACGDHLVVNNSRSSRTARALRHHKYRKTCKRCR
VSDEDLNKFLTKANEDQTSVKVKVVSAPTRTKKAMPKSVARAPKPLENTEAAQAQPSGSKFSPAIPVSTQESV
SVPASVSTSISSISTGATASALVKGNTNPITSMSAPVQASAPALTKSQTDRLEVLLNPKDEISLNSGKPFREL
ESELLSRRKKDLQQIYAEERENYLGKLEREITRFFVDRGFLEIKSPILIPLEYIERMGIDNDTELSKQIFRVD
KNFCLRPMVAPNIFNYARKLDRALPDPIKIFEIGPCYRKESDGKEHLEEFTMLNFFQMGSGCTRENLESIITD
FLNHLGIDFKIVGDSCMVYGDTLDVMHGDLELSSAVVGPIPLDREWGIDKPWIGAGFGLERLLKVKHDFKNIK
RAARSESYYNGISTNL
>Mm_PCKRS/1-454
MDKKPLNTLISATGLWMSRTGTIHKIKHHEVSRSKIYIEMACGDHLVVNNSRSSRTARALRHHKYRKTCKRCR
VSDEDLNKFLTKANEDQTSVKVKVVSAPTRTKKAMPKSVARAPKPLENTEAAQAQPSGSKFSPAIPVSTQESV
SVPASVSTSISSISTGATASALVKGNTNPITSMSAPVQASAPALTKSQTDRLEVLLNPKDEISLNSGKPFREL
ESELLSRRKKDLQQIYAEERENYLGKLEREITRFFVDRGFLEIKSPILIPLEYIERFGIDNDTELSKQIFRVD
KNFCLRPMLSPNLCNYMRKLDRALPDPIKIFEIGPCYRKESDGKEHLEEFTMLNFCQMGSGCTRENLESIITD
FLNHLGIDFKIVGDSCMVYGDTLDVMHGDLELSSAVVGPIPLDREWGIDKPWIGAGFGLERLLKVKHDFKNIK
RAARSESYYNGISTNL

The same principle applies equally to other mutations and/or to other backbones.

Transplanted polypeptides produced in this manner should advantageously be tested to ensure that the desired function/substrate specificities have been preserved.

### BRIEF DESCRIPTION OF THE FIGURES

FIG. 1 depicts strategies for encoding lysine methylation. A. amino acids B. schemes for encoding amino acid (3) of FIG. 1A.

FIG. 2 depicts that amino acid 2 can be site-specifically incorporated into recombinant proteins in response to an amber codon and quantitatively, post-translationally converted to amino acid 3. 2A. Myoglobin-His6 is purified from E. coli containing pMyo4TAGPylT-his6, and pBKPylS in the presence of amino acids 1 or 2 2B. Synthesis of H3K9mel, lane 3, His6 H3 incorporating 2 in place of K9 and deprotected with 2% TFA, lanes 4 and 5 are post-cleavage of the N-terminal His6 tag with TEV protease. 2C. HP1 specifically recognizes H3K9mel. HP1 was used to immunoprecipitate H3 or H3K9mel. The immunoprecipitation was probed for H3 using an anti H3 antibody. Input: 2% of total Histone H3. PD “pull down”. Mock: no HP1 added.

FIG. 3 shows schemes for encoding amino acid (3) of FIG. 1A.

FIG. 4 depicts synthesis a H-Lys(Boc)(Me)-OH (2) from amino acid (5).

FIG. 5 depicts synthesis a H-Lys(Boc)(Me)-OH (2) from amino acid (3).

FIG. 6 shows NMR Spectra. 6A. 1H-NMR of amino acid (2). 6B. 13H-NMR of amino acid (2).

FIG. 7 shows electrospray Ionization Mass Spec (ESI-MS) spectra of myoglobin-His6 demonstrates the quantitative incorporation of amino acid (2) (FIG. 7A). 7B ESI-MS analysis of the purified histone confirms the incorporation of 2 into histone H3. 7C ESI-MS spectra of the deprotected H3K9-2 sample demonstrates that the auxiliary is quantitatively removed under these conditions to reveal N-methyl-L-lysine. 7D MS/MS protein sequencing further confirms that the site of lysine methylation is as genetically encoded.

FIG. 8 shows a photograph of Gel depicting that H3K9mel can be assembled into nucleosomes in vitro with a comparable efficiency to unmodified H3.

FIG. 9 shows alignment of PylS sequences.

FIG. 10 shows sequence identity of PylS sequences.

FIG. 11 shows alignment of the catalytic domain of PylS sequences (from 350 to 480; numbering from alignment of FIG. 9).

FIG. 12 shows sequence identity of the catalytic domains of PylS sequences.

FIG. 13 shows alignment of synthetases with transplanted mutations based on M. barkeri PylS or M. mazei PylS. The red asterisks indicate the mutated positions.

The invention is now described by way of example. These examples are intended to be illustrative, and are not intended to limit the appended claims.

### EXAMPLES

### Example 1

### Production of Polypeptide Comprising N^{ε}-Methyl-Lysine

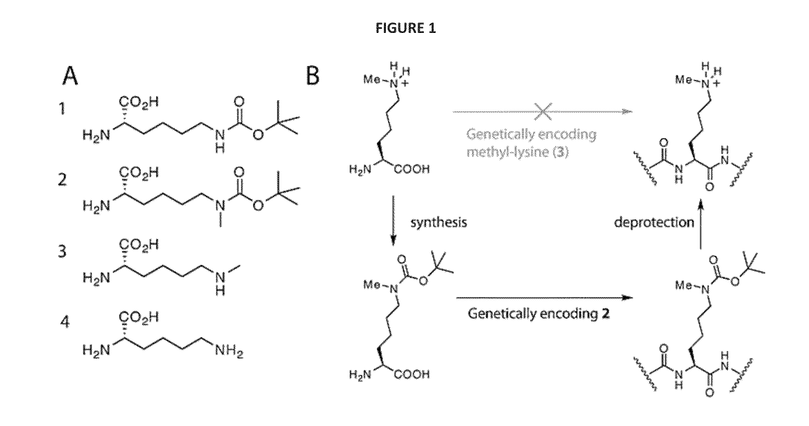

We realized that we might be able to encode N^{ε}-methyl-L-lysine (3) indirectly by providing the synthetase enzyme with a substrate that was significantly different from both N^{ε}-methyl-L-lysine and L-lysine if we were able to subsequently effect the facile, quantitative and specific post-translational conversion of this precursor to N^{ε}-methyl-L-lysine on the synthesized protein. Since N^{ε}-tert-butyl-oxycarbonyl-L-lysine (1) is an efficient substrate for the pyrrolysyl-tRNA synthetase/tRNA_{CUA} pair¹⁹ we asked whether N^{ε}-methyl-L-lysine (3) could be incorporated into proteins in a two-step process in which N^{ε}-tert-butyl-oxycarbonyl-N^{ε}-methyl-L-lysine (2) is genetically incorporated into proteins and the tert-butyl-oxycarbonyl group is removed post-translationally to reveal N^{ε}-methyl-L-lysine (see FIG. 1—Strategies for encoding lysine methylation. A. amino acids used B. Schemes for encoding 3 in recombinant proteins.)

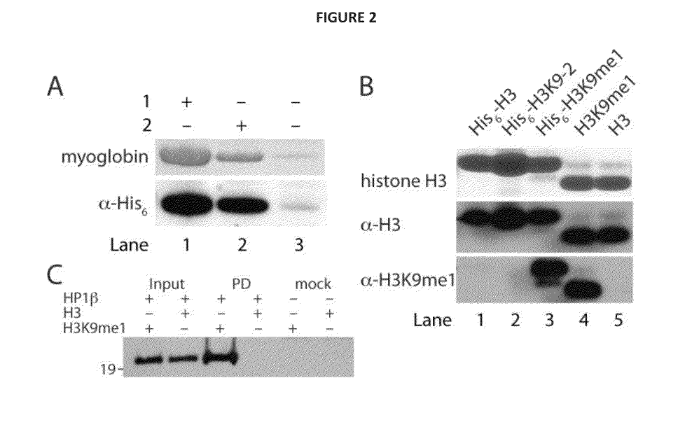

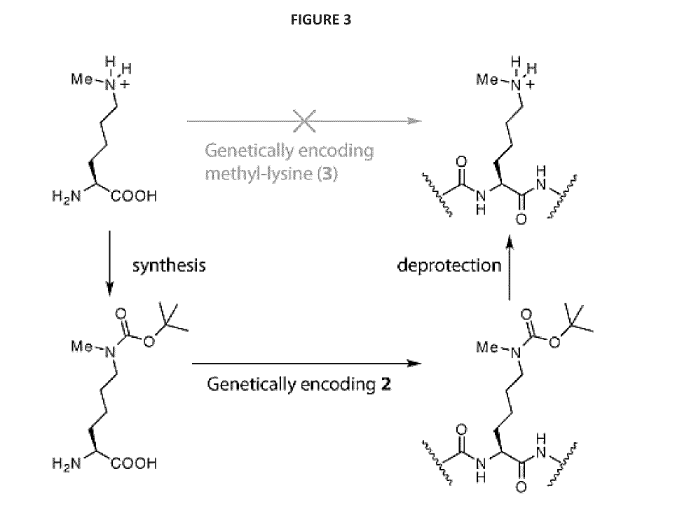

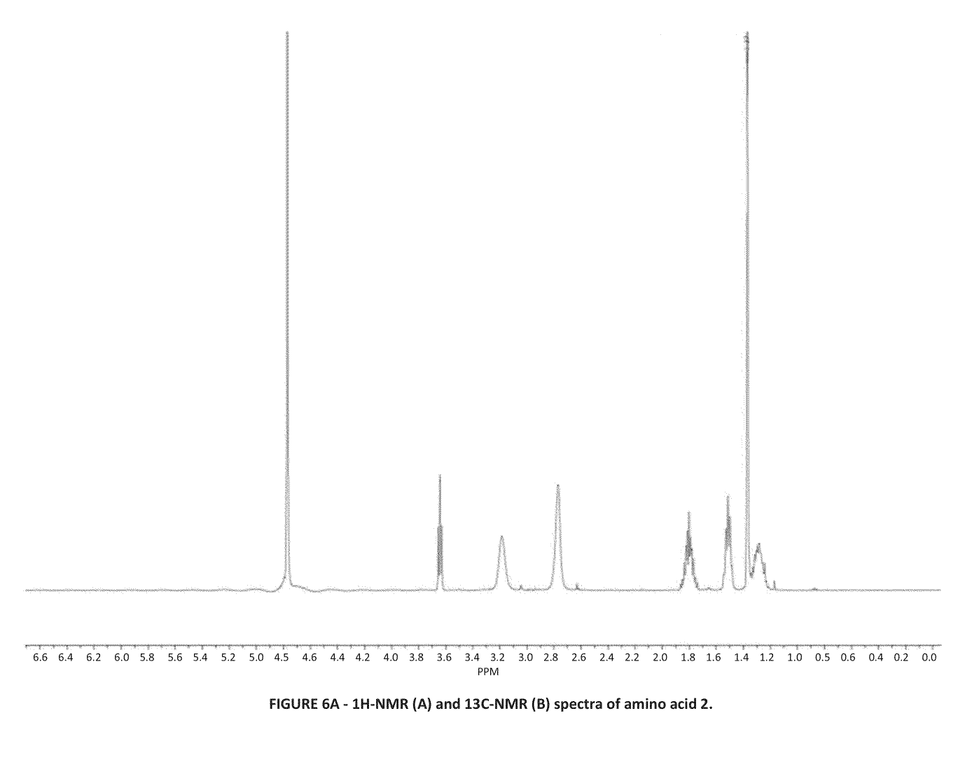

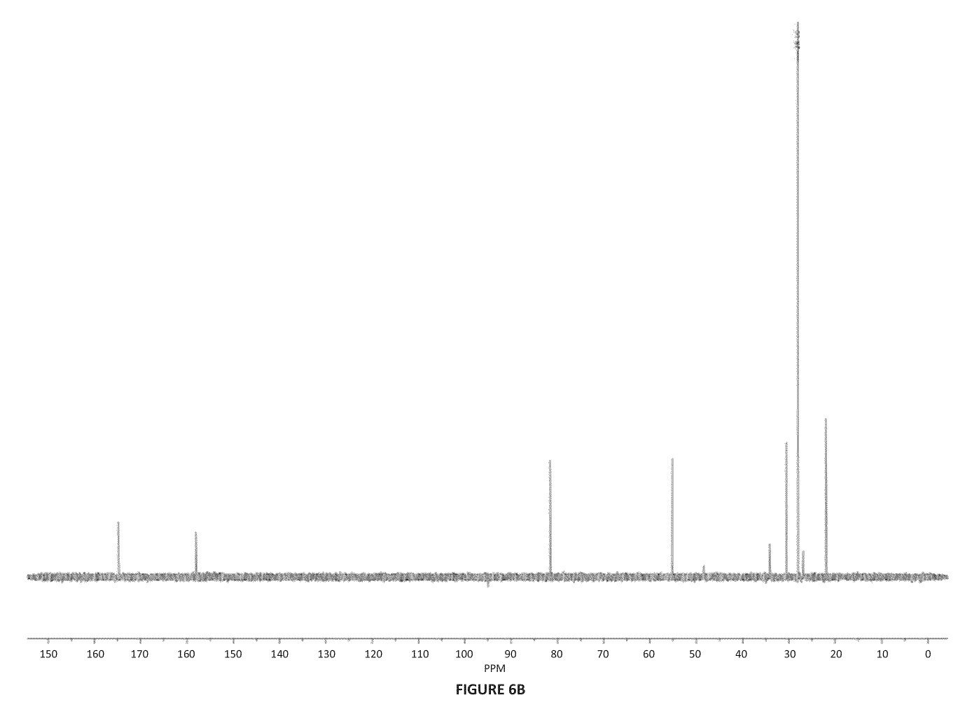

To investigate whether 2 can be incorporated using the pyrrolysyl-tRNA synthetase/tRNA_{CUA} pair we prepared the amino acid in 95% yield by deprotection of commercially available N^{α}-Fmoc-N^{ε}-tert-butyl-oxycarbonyl-N^{ε}-methyl-L-lysine 5. In addition we directly synthesized 2 from 3 in 77% yield (Supplementary schemes 1 & 2, Supplementary methods & FIG. 6). We transformed E. coli with pBKPylS (which encodes the Methanosarcina barkeri pyrrolysyl-tRNA synthetase, MbPylRS) and pMyo4TAGPylT-his₆ (which encodes MbtRNA_{CUA} and a C-terminally hexahistidine tagged sperm whale myoglobin gene with an amber codon at position 4)¹⁴ and induced protein expression with and without the addition of 2 to mid-log phase cells. Full-length myoglobin was only produced and purified in good yield in the presence of 2 (see FIG. 2: 2 can be site-specifically incorporated into recombinant proteins in response to an amber codon and quantitatively, post-translationally converted to 3. A. Myoglobin-His₆ is purified from E. coli containing pMyo4TAGPylT-his₆, and pBKPylS in the presence of amino acids 1 or 2 B. Synthesis of H3K9me1, lane 3, His₆ H3 incorporating 2 in place of K9 and deprotected with 2% TFA, lanes 4 and 5 are post-cleavage of the N-terminal His₆ tag with TEV protease. C. HP1 specifically recognizes H3K9me1. HP1 was used to immunoprecipitate H3 or H3K9me1. The immunoprecipitation was probed for H3 using an anti H3 antibody. Input: 2% of total Histone H3. PD “pull down”. Mock: no HP1 added).

### Example 2

### MS Analysis

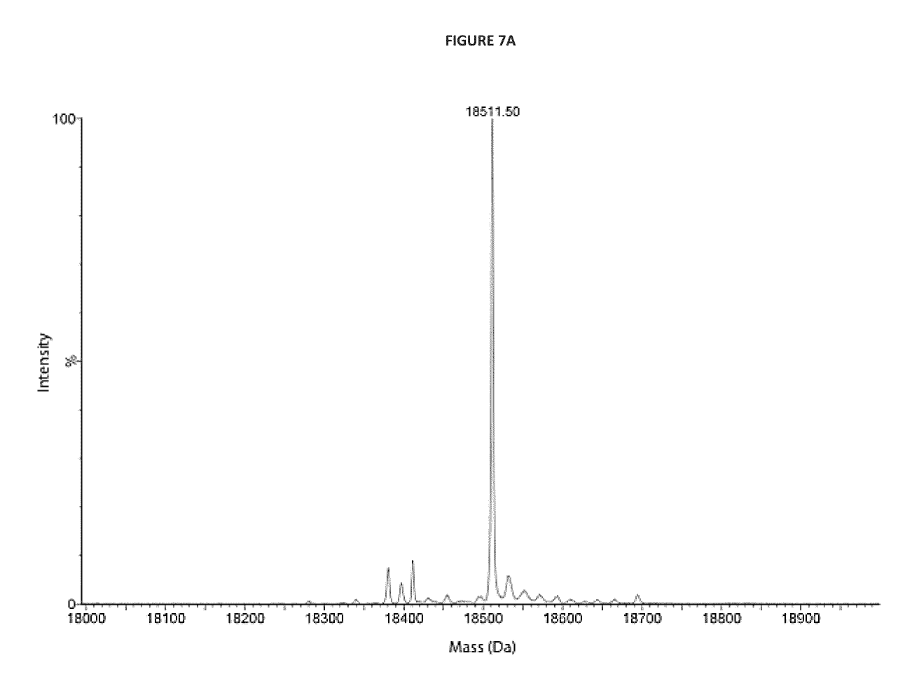

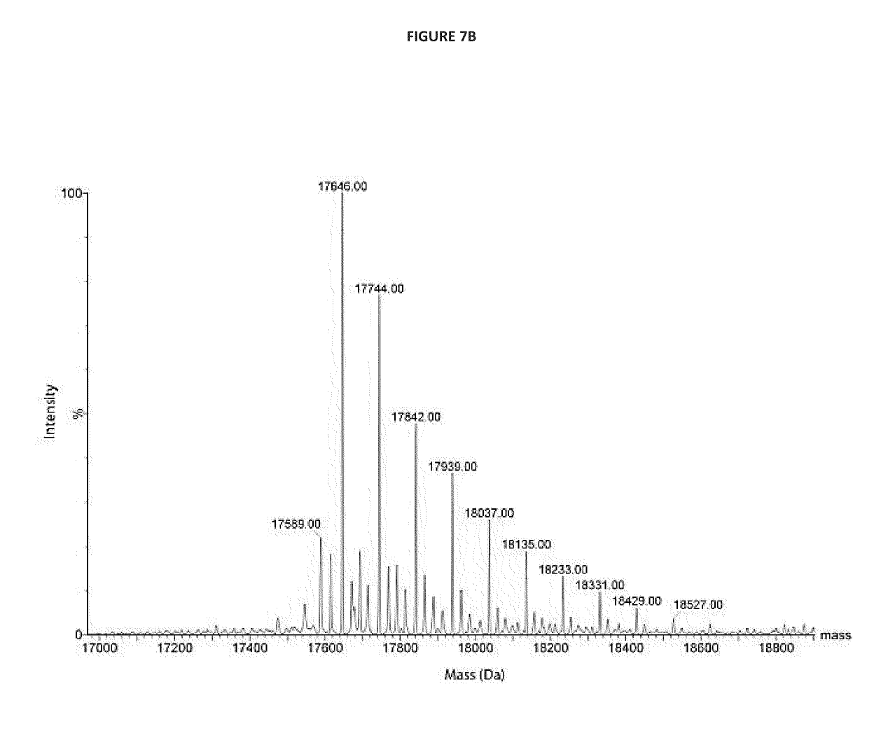

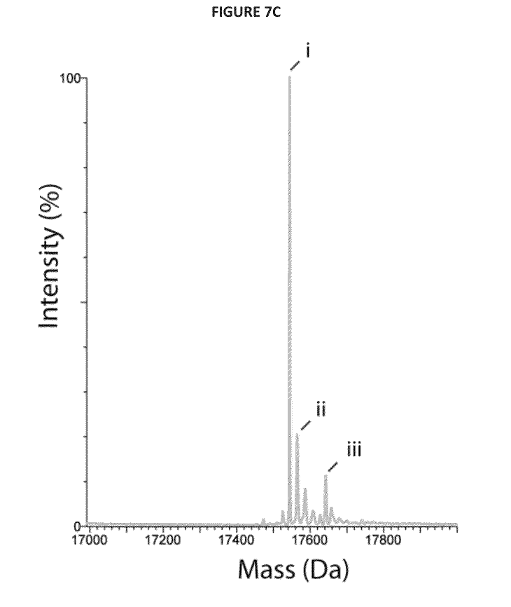

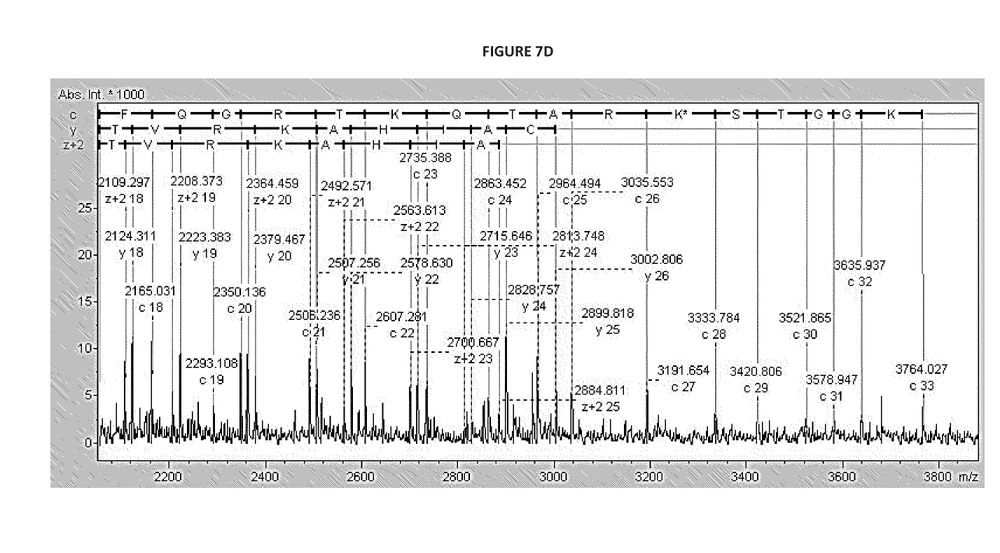

To demonstrate that 2 can be incorporated with high fidelity into recombinant proteins and is not subjected to in vivo modification¹⁴, we performed electrospray ionization mass spectrometry (ESI-MS) on the purified protein. The ESI-MS spectra of myoglobin-His₆ demonstrates the quantitative incorporation of 2 (FIG. 7A). These data demonstrate that 2 can be genetically encoded in proteins in good yield and with high fidelity using MbPylRS/MbtRNA_{CUA} pair.

### Example 3

### Application to Histones

To specifically and efficiently introduce 2 in a histone at physiologically relevant site, we transformed E. coli BL21(DE3) with pBKPylS and pCDF-PylT-H3K9TAG (a vector which encodes MbtRNA_{CUA} and a N-terminally hexahistidine tagged histone H3 gene in which the codon for lysine 9 is replaced with an amber codon)¹⁵. We grew the cells in the presence of 2 mM 2, and expressed and purified the recombinant histone in good yield (2 mg per liter of culture). ESI-MS analysis of the purified histone confirms the incorporation of 2 into histone H3 (FIG. 7B).

### Example 4

### Removal of Auxiliary Group

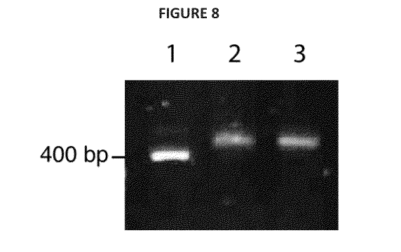

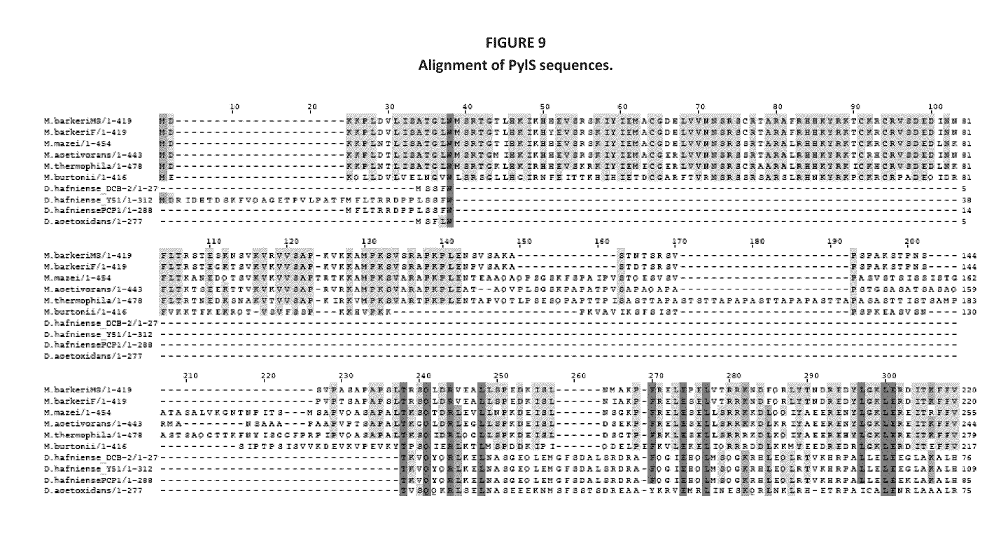

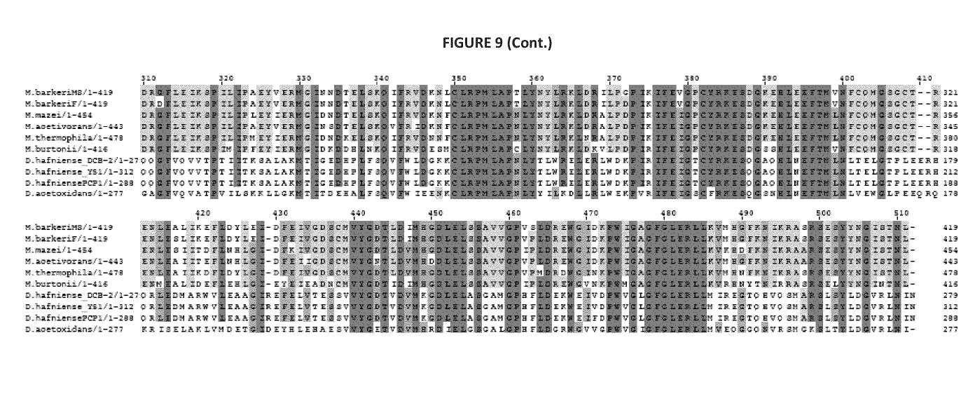

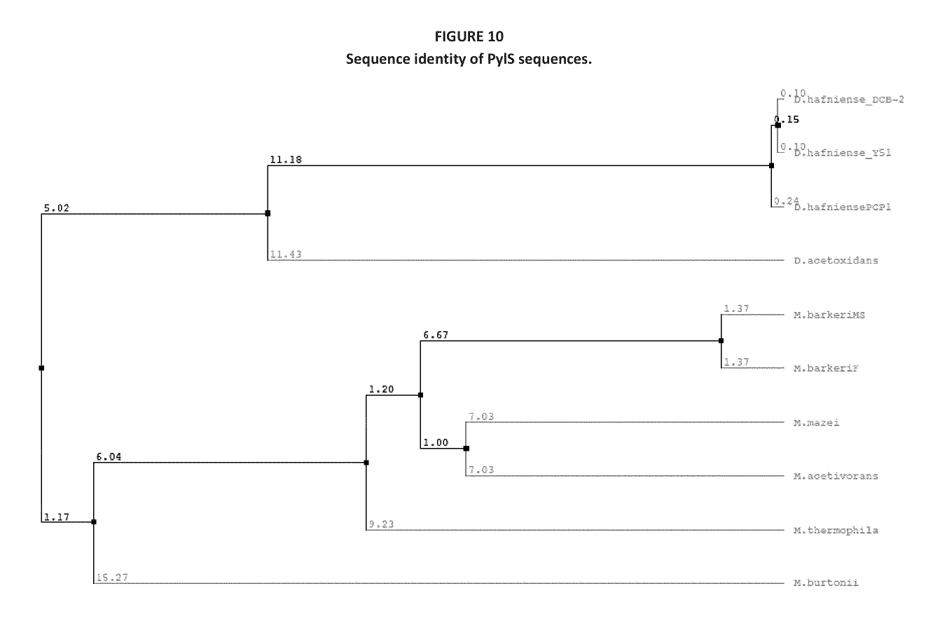

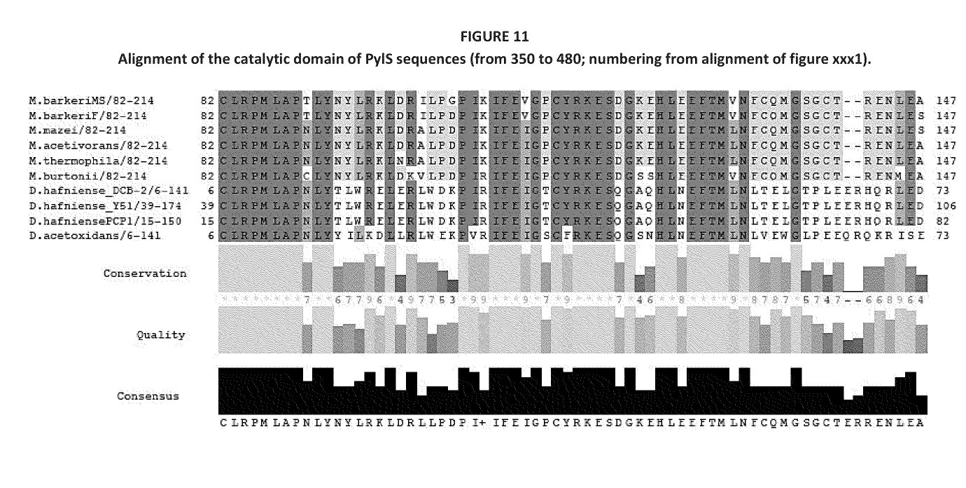

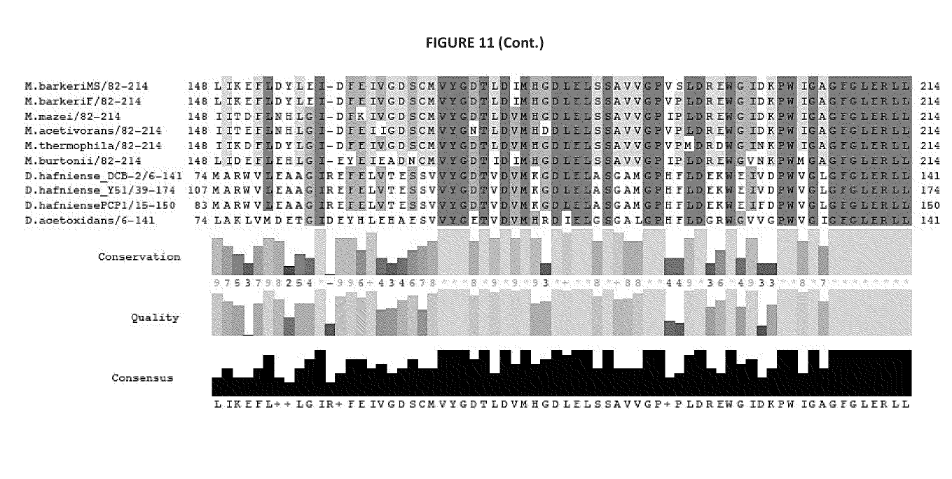

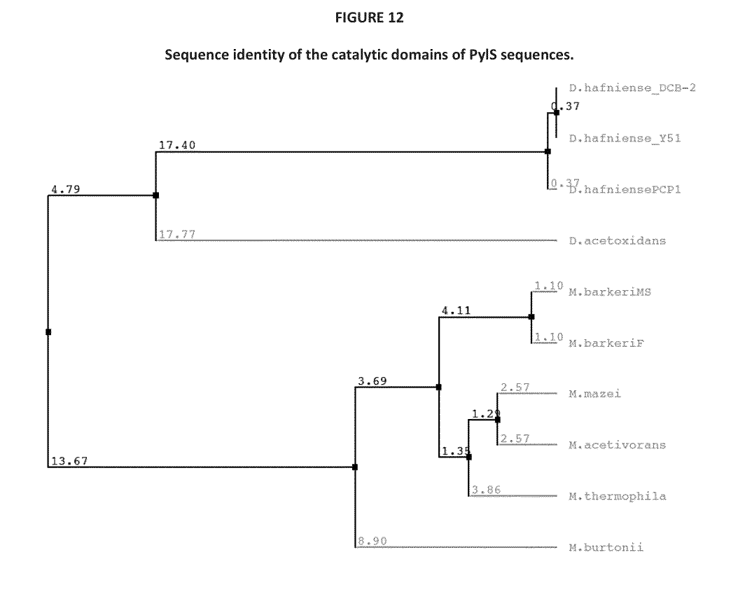

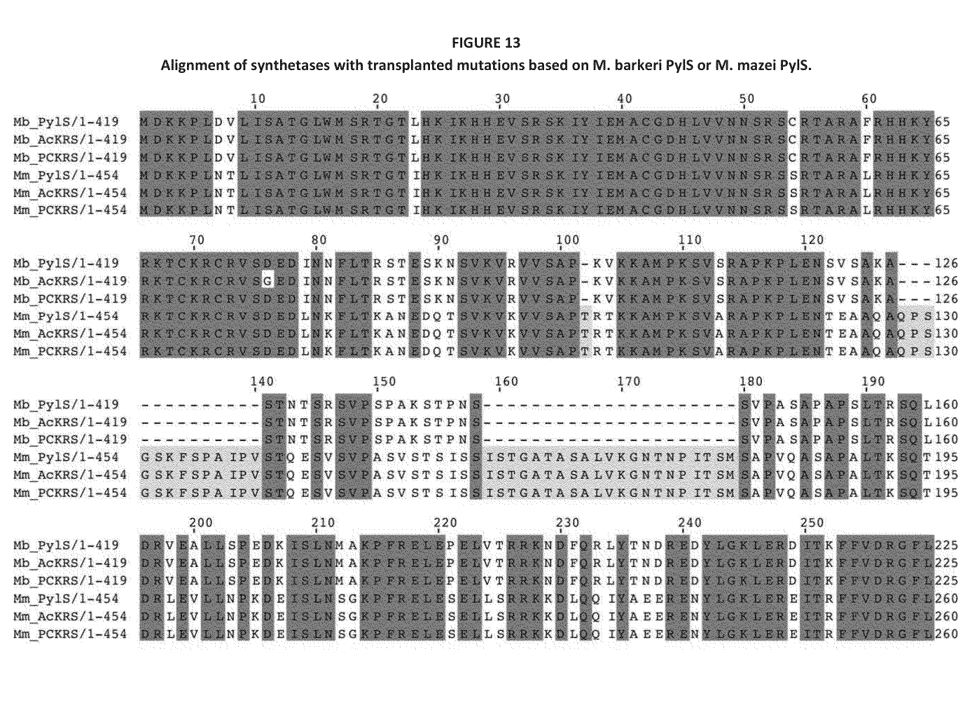

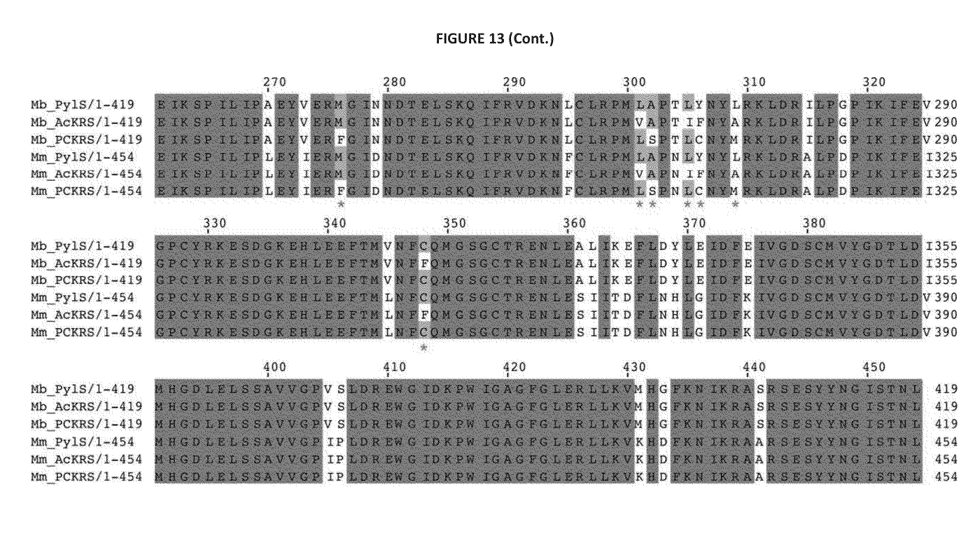

To demonstrate that the tert-butyl-oxycarbonyl group can be quantitatively removed from the histone under mild conditions, the purified H3K9-2 was treated with a solution of 2% trifluoroacetic acid (TFA) for 4 h at 37° C. Western blots with an anti-H3K9me1 antibody against unmodified H3, H3 bearing 2 at position 9 (H3K9-2) and the TFA treated H3K9-2 confirmed the presence of methyl-L-lysine at position 9 in the deprotected sample (FIG. 2, lane 3). The ESI-MS spectra of the deprotected H3K9-2 sample (FIG. 7C) demonstrates that the auxiliary is quantitatively removed under these conditions to reveal N^{ε}-methyl-L-lysine. MS/MS protein sequencing (FIG. 7D) further confirms that the site of lysine methylation is as genetically encoded. H3K9me1 can be assembled into nucleosomes in vitro with a comparable efficiency to unmodified H3 (FIG. 8).

### Example 5

### Biological Functions Retained

To demonstrate the biochemical activity of the methylated histone generated by our approach we performed immunoprecipitations with heterochromatin protein 1 (HP1) (FIG. 2C), a chromodomain protein²⁰ that does not bind to unmethylated H3, but is known to specifically bind to short peptides based on a histone H3 tail bearing mono-, di-, or tri-methylated K9 (with a preference for di- and tri-methylated H3 K9)²¹. HP1 immunoprecipitation of full-length H3K9me1, synthesized by our approach, and full length H3 allows us to demonstrate that HP1 binds specifically to full-length H3K9me1 over unmethylated H3.

### Summary of Examples

In conclusion, we have created a general method for the quantitative, site-specific incorporation of N^{ε}-methyl-L-lysine in recombinant proteins. The method has two steps: first an amino acid containing an auxiliary group is used to differentiate N^{ε}-methyl-L-lysine from L-lysine and to provide a good substrate for the pyrrolysyl synthetase; second the auxiliary group is removed to reveal N^{ε}-methyl-L-lysine. We have demonstrated the utility of the method by site-specifically installing N^{ε}-methyl-L-lysine into full-length histone H3 and demonstrated that the modified H3 specifically recruits HP1²¹. We are currently extending our approach to installing other modifications implicated in the histone code and epigenetic inheritance to understand how combinations of post-translational modifications program cellular outcomes.

FIG. 7. Genetic incorporation of 2 in recombinant myoglobin and recombinant histone H3. (A) ESI-MS analysis of the purified myoglobin-His6 incorporating 2 (Found mass 18511.50±0.50 Da, expected mass 18511.20 Da). (B) ESI-MS analysis of the purified histone His6-H3 incorporating 2 at lysine 9 (Found mass 17646.0±1.0 Da, expected mass 17647.0 Da). Several phosphate adducts each differing by 98 Da are seen in these spectra, as often found for highly basic proteins such as histones. The peak of mass 17589.0 Da corresponds to loss of t-butyl group (−57 Da) during electrospray ionization process. (C) H3K9me1 is produced quantitatively from H3K9-2. ESI-MS analysis of His₆-H3K9me1 after the deprotection of H3K9-2 with 2% trifluoroacetic acid (Found mass 17547.00±0.50 Da, expected mass 17547.10 Da, the minor peaks labeled ii and iii correspond to non-covalent sodium and phosphate adducts, respectively. (D) Top-down sequencing of H3K9-2 after TFA deprotection, confirms the site of H3K9me1 incorporation is as genetically programmed. The purified protein was subjected to MALDI top-down sequencing as described in the supplementary methods. The protein sequence was inferred from the mass differences of individual ions and confirms the site specific-incorporation of methyl-lysine at position 9 of histone H3 (K* has a mass 14 Da greater than observed for lysine). Mass difference of K* to lysine=(c33−c32)−(c28−c27). No peaks are observed corresponding to H3K9-2 or H3K9, further confirming the fidelity of incorporation and the quantitative deprotection under our conditions.

### FIG. 8.

Nucleosome reconstitution in the presence of H3 and H3K9me1. Binding of histone octamers to the 2×200-601 DNA array. Lane 1: purified 2×200-601 DNA. Lane 2 nucleosomes assembled with H3. Lane 3 Nucleosomes assembled with H3K9me1. Samples were analyzed after electrophoresis for 20 minutes at 20 V/cm in a 1% agarose gel buffered with 0.4×TBE and stained with ethidium bromide.

### Supplementary Methods

### Chemical Synthesis

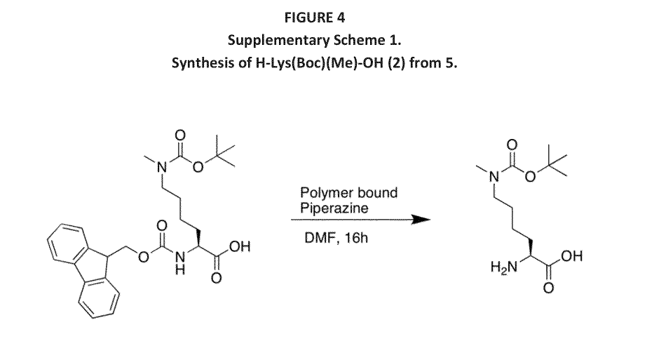

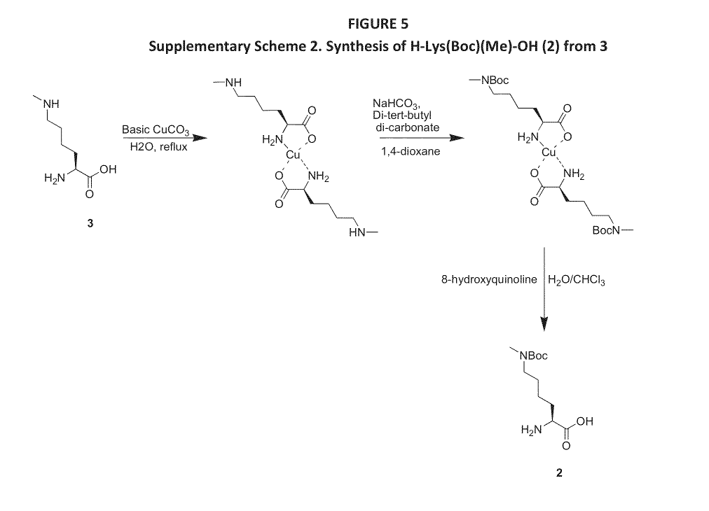

### Please See Supplementary Schemes 1 &2 Shown in FIGS. 4 and 5

### Synthesis of (S)-2-amino-6-(tert-butoxycarbonyl(methyl)amino)hexanoic acid (2) from 5

Polymer bound piperazine (loading 1.5 mmol/g) (1.66 g, ˜2.49 mmol) was added to a stirred solution of Fmoc-Lys (Boc) (Me)-OH (5) (0.8 g, 1.66 mmol, Bachem) in dry DMF (10 mL). The resulting reaction mixture was stirred at room temperature for 16 h. The suspension was filtered through a sintered funnel and washed with distilled water (˜40 mL). Water was removed by lyophilization to give (H-Lys (Boc) (Me)-OH) (2) as a white solid (4.1 g, 1.57 mmol, 95%) mp: 215-216° C.

HRMS (ESI⁺) m/z found 283.1642 [M+Na]⁺C₁₂H₂₄N₂NaO₄⁺ required 283.1634

¹H NMR (500 MHz, D₂O) δ 3.64 (t, J=6.2, 1H), 3.19 (s, 2H), 2.77 (s, 3H), 1.89-1.72 (m, 2H), 1.58-1.45 (m, 2H), 1.36 (s, 9H), 1.35-1.19 (m, 2H).

¹³C NMR (126 MHz, CDCl₃) δ 174.83, 158.04, 81.58, 55.18, 48.41, 34.14, 30.58, 28.10, 26.92, 22.01.

### Synthesis of (S)-2-amino-6-(tert-butoxycarbonyl(methyl)amino)hexanoic acid (2) from 3

To a solution of H-Lys (Me)-OH.HCl (1 g, 5.1 mmol) in water (7 mL) basic CuCO₃ (i.e. CuCO₃.Cu(OH)₂.H₂O, 0.752 g, 3.4 mmol) was added. The resulting mixture was heated under reflux for 30 min and the hot solution was filtered trough Celite. The filter pad was washed with hot water (˜20 mL). The filtrate was cooled to 10° C. and basified with NaHCO₃ (0.857 g, 10.20 mmol). Di-tert-butyl di-carbonate (1.48 g, 6.8 mmol) in dioxane (12 mL) was added to this solution and stirred for 16 h. Dioxane was removed under reduced pressure 8-hydroxyquinoline in chloroform (25 mL) was added to the resulting solution. After stirring for 3 h, reaction mixture was filtered through sintered funnel and washed with water (10 mL). The chloroform layer was separated and the water layer was extracted with chloroform (3×25 mL) and neutralized by 0.5 N HCl. Water was removed by lyophilization and the resulting solid was dissolved in methanol/dichloromethane (1:4, 50 mL). Insoluble salt was removed by filtration and the filtrate was evaporated to give H-Lys (Boc) (Me)-OH as a white solid (1 g, 3.84 mmol, 77%)

HRMS (ESI⁺) m/z found 283.1642 [M+Na]⁺C₁₂H₂₄N₂NaO₄⁺ required 283.1634

¹H NMR (500 MHz, D₂O) δ 3.64 (t, J=6.2, 1H), 3.19 (s, 2H), 2.77 (s, 3H), 1.89-1.72 (m, 2H), 1.58-1.45 (m, 2H), 1.36 (s, 9H), 1.35-1.19 (m, 2H).

¹³C NMR (126 MHz, CDCl₃) δ 174.83, 158.04, 81.58, 55.18, 48.41, 34.14, 30.58, 28.10, 26.92, 22.01.

### Protein Expression and Purification

To express sperm whale myoglobin incorporating unnatural amino acids (Neumann, H.; Peak-Chew, S. Y.; Chin, J. W., Nat Chem Biol 2008, 4, 232-4) we transformed E. coli DH10B cells with pBKPylS and pMyo4TAGPylT-his₆. Cells were recovered in 1 mL of LB media for 1 h at 37° C., before incubation (16 h, 37° C., 250 r.p.m.) in 100 mL of LB containing kanamycin (50 μg/mL) and tetracycline (25 μg/mL). 10 mL of this overnight culture was used to inoculate 250 mL of LB supplemented with kanamycin (25 μg/mL), tetracycline (12 μg/mL) and 3 mM of 2. Cells were grown (37° C., 250 r.p.m.), and protein expression was induced at OD600 ˜0.6, by addition of arabinose to a final concentration of 0.2%. After 3 h of induction, cells were harvested. Proteins were extracted by sonication at 4° C. The extract was clarified by centrifugation (20 mM, 21,000 g, 4° C.), 1 mL of 50% Ni²⁺-NTA beads (Qiagen) were added to the extract, the mixture was incubated with agitation for 1 h at 4° C. Beads were collected by centrifugation (10 min, 1000 g). The beads were twice resuspended in 50 mL wash buffer and spun down at 1000 g. Subsequently, the beads were resuspended in 20 mL of wash buffer and transferred to a column. Protein was eluted in 1 mL of wash buffer supplemented with 200 mM imidazole and was then re-buffered to 20 mM ammonium bicarbonate using a sephadex G25 column. The purified proteins were analysed by 4-12% SDS-PAGE.

To express histone H3 with an incorporated unnatural amino acid, we transformed E. coli B121(DE3) cells with pBKPylS and pCDF-PylT-H3K9TAG (which encodes histone H3 bearing an amber codon at position 9 and an N-terminal His₆-tag followed by a TEV protease cleavage site sequence, as well as MbtRNA_{CUA} on an lpp promoter and rrnC terminator. The plasmid has a spectinomycin resistance marker). Cells were recovered in 1 mL of SOC media for 1 h at 37° C., before incubation (16 h, 37° C., 250 r.p.m.) in 100 mL of 2×TY containing kanamycin (50 μg/mL) and spectinomycin (70 μg/mL). 25 mL of this overnight culture was used to inoculate 500 mL of 2×TY supplemented with kanamycin (25 μg/mL), spectinomycin (35 μg/mL) and 2 mM of 2. Cells were grown (37° C., 250 r.p.m.), and protein expression was induced at OD600 ˜0.9, by addition of IPTG to a final concentration of 1 mM. After 5 h of induction, cells were harvested and resuspended in 50 mL of 1×PBS containing 1 mM DTT, lysozyme (1 mg/mL), DNaseI (100 μg/mL), 1 mM PMSF, and Roche protease inhibitor cocktail. The cells were disrupted by sonication. The cell lysates were centrifuged at 17,000 rpm for 20 min at 4° C. The supernatant was discarded and the pellet was retained as the insoluble fraction. The pellet was resuspended in 25 mL of 1×PBS supplemented with 1 mM DTT and 1% Triton-X, and centrifuged at 17,000 rpm for 20 min at 4° C. The pellet was subsequently resuspended in 25 mL of 1×PBS containing 1 mM DTT, and centrifuged at 17,000 rpm for 20 mM at 4° C. The insoluble fraction was incubated in 350 μL of DMSO for 30 min at room temperature, and dissolved in 25 mL of 20 mM Tris-HCl buffer (pH 8.0) containing 6 M guanidinium chloride and 1 mM DTT. The solution was incubated with vigorous shaking at 37° C. for 1 h and centrifuged at 17,000 rpm for 20 min at 4° C. The supernatant was equilibrated with 1 mL of 50% Ni-NTA beads (Qiagen) for 1 h at room temperature. The beads were collected by centrifugation at 2,400 rpm for 5 mM. The beads were washed with 15 mL of 100 mM sodium phosphate buffer (pH 6.2) containing 8 M urea and 1 M DTT. The protein was eluted with 20 mM sodium acetate buffer (pH 4.5) supplemented with 7 M urea, 200 mM NaCl and 1 mM DTT in 500 μL fractions. The fractions of the purified proteins were analysed by 4-12% SDS-PAGE. The protein-containing fractions were combined, dialyzed overnight in 1 mM DTT solution and stored at −20° C.

Heterochromatin protein 1 homolog beta (HP1b) from mouse, cloned into pET-16 (Novagen) expression vector was expressed in E. coli C41(DE3) and purified by Ni-affinity, anion exchange chromatography and gel filtration.

### Preparation of Monomethylated Histones

The protein H3K9-2 (40 nmol) was incubated with shaking (800 rpm) in 1 mL of 1% TFA for 4 h at 37° C. to produce H3K9me1. The protein was rebuffered to 1 mM DTT (1.5 mL) using a sephadex G25 column. The hexahistidine tag was removed by incubating with TEV protease (1.5 mg/mL, 100 μL) in 50 mM Tris buffer (pH 7.4) for 5 h at 30° C. and overnight dialysis in 1 mM DTT.

### Immunoprecipitation of Full-Length H3 and H3K9me1 by HP1

HP1β (1 μM) was incubated with H3 histone or H3K9me1 histone, in 600 μl of binding buffer (0.5 M NaCl, 1% NP40, 0.5% sodium deoxycholate, 0.1% SDS, 50 mM Tris HCl pH 8.0). 10 μl of this sample was removed to check total protein levels (input). The remaining supernatant was incubated for 4 h at 4° C. with 1 μg of a goat polyclonal antibody to CBX1/HP1 beta (Abcam, ab40828). After one hour of incubation 30 μl of protein A-agarose (Sigma) was added. The beads were pelleted, washed 5 times with 700 μl RIPA buffer, and bound protein was eluted by boiling in SDS-sample buffer. A Rabbit polyclonal antibody to C-terminus of H3 (9715, Cell Signaling Technology) was used to detect H3 proteins immunoprecipitated by HP1.

### Protein Mass Spectrometry

Protein total mass was determined on an LCT time-of-flight mass spectrometer with electrospray ionization (ESI, Micromass). Proteins were rebuffered in 20 mM of ammonium bicarbonate and mixed 1:1 with formic acid (1% in methanol/H2O=1:1). Samples were injected at 10 μl min⁻¹ and calibration was performed in positive ion mode using horse heart myoglobin. 60 scans were averaged and molecular masses obtained by deconvoluting multiply charged protein mass spectra using MassLynx version 4.1 (Micromass). Theoretical masses of wild-type proteins were calculated using Protparam (http://us.expasy.org/tools/protparam.html), and theoretical masses for unnatural amino acid containing proteins were adjusted manually. Where indicated methylation position sequencing was performed using a top down approach, in these cases in-source decay (ISD) spectra were acquired in reflectron mode on an Ultraflex III TOF/TOF mass spectrometer (Bruker Daltonics, Bremen, Germany) using a 2,5-dihydroxy benzoic acid matrix.

### Nucleosome Reconstitution

Xenopus H4, H2A, and H2B were expressed and purified as described (Neumann, H.; Hancock, S.; Buning, R.; Routh, A.; Chapman, L.; Somers, J.; Owen-Hughes, T.; van Noort, J.; Rhodes, D.; Chin, J. W., 2009, in press). Octamer reconstitution was carried out in 2M NaCl, 10 mM TE (pH 7.4), 1 mM EDTA (pH 7.4) and reconstituted octamers purified by gel filtration. The DNA 601 repeat fragment cloned in pUC18 vector was digested with EcoRV and purified by PEG precipitation. Nucleosome reconstitutions were carried by addition of 40 ng of 400 bp DNA molecules containing two copies of the 601 repeat. Nucleosomes were assembled by a continuous dialysis method in which the NaCl concentration was reduced from 2.0 M to 10 mM over a 24 hour period at 4° C. (Huynh, V. A.; Robinson, P. J.; Rhodes, D., J Mol Biol 2005, 345, 957-68). Nucleosome assembly was tested using gel mobility-shift assays in 0.7%-1% (w/v) agarose gels run in 0.4×TBE.

### REFERENCES

1. Kohler, C.; Villar, C. B., Trends Cell Biol 2008, 18, 236-43.
2. Spivakov, M.; Fisher, A. G., Nat Rev Genet 2007, 8, 263-71.
3. Martin, C.; Zhang, Y., Nat Rev Mol Cell Biol 2005, 6, 838-49.
4. Strahl, B. D.; Allis, C. D., Nature 2000, 403, 41-5.
5. Martino, F.; Kueng, S.; Robinson, P.; Tsai-Pflugfelder, M.; van Leeuwen, F.; Ziegler, M.; Cubizolles, F.; Cockell, M. M.; Rhodes, D.; Gasser, S. M., Mol Cell 2009, 33, 323-34.
6. Shogren-Knaak, M.; Ishii, H.; Sun, J. M.; Pazin, M. J.; Davie, J. R.; Peterson, C. L., Science 2006, 311, 844-7.
7. He, S.; Bauman, D.; Davis, J. S.; Loyola, A.; Nishioka, K.; Gronlund, J. L.; Reinberg, D.; Meng, F.; Kelleher, N.; McCafferty, D. G., Proc Natl Acad Sci USA 2003, 100, 12033-8.
8. Shogren-Knaak, M. A.; Fry, C. J.; Peterson, C. L., J. Biol. Chem. 2003, 278, 15744-15748.
9. McGinty, R. K.; Kim, J.; Chatterjee, C.; Roeder, R. G.; Muir, T. W., Nature 2008, 453, 812-6.
10. Guo, J.; Wang, J.; Lee, J. S.; Schultz, P. G., Angew Chem Int Ed Engl 2008, 47, 6399-401.
11. Simon, M. D.; Chu, F.; Racki, L. R.; de la Cruz, C. C.; Burlingame, A. L.; Panning, B.; Narlikar, G. J.; Shokat, K. M., Cell 2007, 128, 1003-12.
12. Gellman, S. H., Biochemistry 1991, 30, 6633-6.
13. Gloss, L. M.; Kirsch, J. F., Biochemistry 1995, 34, 3990-8.
14. Neumann, H.; Peak-Chew, S. Y.; Chin, J. W., Nat Chem Biol 2008, 4, 232-4.
15. Neumann, H.; Hancock, S.; Buning, R.; Routh, A.; Chapman, L.; Somers, J.; Owen-Hughes, T.; van Noort, J.; Rhodes, D.; Chin, J. W., 2009, in preparation.
16. Fersht, A. R., Biochemistry 1977, 16, 1025-30.
17. Nureki, O.; Vassylyev, D. G.; Tateno, M.; Shimada, A.; Nakama, T.; Fukai, S.; Konno, M.; Hendrickson, T. L.; Schimmel, P.; Yokoyama, S., Science 1998, 280, 578-82.
18. Polycarpo, C. R.; Herring, S.; Berube, A.; Wood, J. L.; Soll, D.; Ambrogelly, A., FEBS Lett 2006, 580, 6695-700.
19. Mukai, T.; Kobayashi, T.; Hino, N.; Yanagisawa, T.; Sakamoto, K.; Yokoyama, S., Biochem Biophys Res Commun 2008, 371, 818-22.
20. Kim, J.; Daniel, J.; Espejo, A.; Lake, A.; Krishna, M.; Xia, L.; Zhang, Y.; Bedford, M. T., EMBO Rep 2006, 7, 397-403.
21. Fischle, W.; Wang, Y.; Jacobs, S. A.; Kim, Y.; Allis, C. D.; Khorasanizadeh, S., Genes Dev 2003, 17, 1870-81.

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method of making a polypeptide comprising at least one N^{ε}-methyl-lysine at a specific site in said polypeptide, said method comprising
(a) genetically directing the incorporation of R—N^{ε}-methyl-lysine into said polypeptide, wherein R comprises an auxiliary group; and
(b) catalysing the removal of R from the polypeptide of (a).

2. A method according to claim 1 wherein genetically directing the incorporation of R—N^{ε}-methyl-lysine into said polypeptide comprises arranging for the translation of a RNA encoding said polypeptide, wherein said RNA comprises an amber codon, and wherein said translation is carried out in the presence of an amber tRNA charged with R—N^{ε}-methyl-lysine.

3. A method according to claim 2 wherein the tRNA charged with R—N^{ε}-methyl-lysine is supplied by providing a combination of tRNA capable of being charged with R—N^{ε}-methyl-lysine, a tRNA synthetase capable of charging said tRNA with R—N^{ε}-methyl-lysine, and R—N^{ε}-methyl-lysine.

4. A method according to claim 2 wherein the tRNA synthetase capable of charging said tRNA with R—N^{ε}-methyl-lysine comprises Methanosarcina barkeri pyrrolysyl-tRNA synthetase (MBPylRS).

5. A method according to claim 2 wherein the tRNA capable of being charged with R—N^{ε}-methyl-lysine comprises Methanosarcina barkeri tRNA_{CUA}.

6. A method according to claim 1 wherein R comprises tert-butyl-oxycarbonyl.

7. A method according to claim 1 wherein removal of R from the polypeptide comprises treatment of the polypeptide with 2% trifluoroacetic acid (TFA) for 4 hours at 37° C.

8. A method according to claim 1 wherein the polypeptide comprises a histone.

9. A method according to claim 8 wherein the histone is core histone H3.

10. A method according to claim 9 wherein the histone is methylated specifically at residue 9.

11. A method of monitoring DNA breathing comprising (i) providing a histone polypeptide produced according to claim 1 and (ii) measuring the ability of the histone to bind to heterochromatin protein 1.

12. A method to determine the effect of a modulator of DNA breathing which comprises the following steps:
i) providing two samples of histone polypeptide produced according to claim 8,
ii) introducing the modulator to one of said samples,
iii) measuring the ability of the histone to bind to heterochromatin protein 1; wherein if the histone polypeptide sample of (ii) binds to heterochromatin protein 1 less than the second histone polypeptide sample, then it is determined that the modulator has a dampening effect on DNA breathing.


SEQUENCE LISTING (14 sequences)

SEQ ID NO: 1 - protein, 419 aa, Methanosarcina barkeri
MDKKPLDVLISATGLWMSRTGTLHKIKHYEVSRSKIYIEMACGDHLVVNNSRSCRTARAFRHHKYRKTCKRCRVSDEDINNFLTRSTEGKTSVKVKVVSAPKVKKAMPKSVSRAPKPLENPVSAKASTDTSRSVPSPAKSTPNSPVPTSAPAPSLTRSQLDRVEALLSPEDKISLNIAKPFRELESELVTRRKNDFQRLYTNDREDYLGKLERDITKFFVDRDFLEIKSPILIPAEYVERMGINNDTELSKQIFRVDKNLCLRPMLAPTLYNYLRKLDRILPDPIKIFEVGPCYRKESDGKEHLEEFTMVNFCQMGSGCTRENLESLIKEFLDYLEIDFEIVGDSCMVYGDTLDIMHGDLELSSAVVGPVPLDREWGIDKPWIGAGFGLERLLKVMHGFKNIKRASRSESYYNGISTNL

SEQ ID NO: 2 - protein, 419 aa, Methanosarcina barkeri; identical to SEQ ID NO: 1

SEQ ID NO: 3 - protein, 454 aa, Methanosarcina mazeii
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMLAPNLYNYLRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 4 - protein, 443 aa, Methanosarcina acetivorans
MDKKPLDTLISATGLWMSRTGMIHKIKHHEVSRSKIYIEMACGERLVVNNSRSSRTARALRHHKYRKTCRHCRVSDEDINNFLTKTSEEKTTVKVKVVSAPRVRKAMPKSVARAPKPLEATAQVPLSGSKPAPATPVSAPAQAPAPSTGSASATSASAQRMANSAAAPAAPVPTSAPALTKGQLDRLEGLLSPKDEISLDSEKPFRELESELLSRRKKDLKRIYAEERENYLGKLEREITKFFVDRGFLEIKSPILIPAEYVERMGINSDTELSKQVFRIDKNFCLRPMLAPNLYNYLRKLDRALPDPIKIFEIGPCYRKESDGKEHLEEFTMLNFCQMGSGCTRENLEAIITEFLNHLGIDFEIIGDSCMVYGNTLDVMHDDLELSSAVVGPVPLDREWGIDKPWIGAGFGLERLLKVMHGFKNIKRAARSESYYNGISTNL

SEQ ID NO: 5 - protein, 478 aa, Methanosarcina thermophila
MDKKPLNTLISATGLWMSRTGKLHKIRHHEVSKRKIYIEMECGERLVVNNSRSCRAARALRHHKYRKICKHCRVSDEDLNKFLTRTNEDKSNAKVTVVSAPKIRKVMPKSVARTPKPLENTAPVQTLPSESQPAPTTPISASTTAPASTSTTAPAPASTTAPAPASTTAPASASTTISTSAMPASTSAQGTTKFNYISGGFPRPIPVQASAPALTKSQIDRLQGLLSPKDEISLDSGTPFRKLESELLSRRRKDLKQIYAEEREHYLGKLEREITKFFVDRGFLEIKSPILIPMEYIERMGIDNDKELSKQIFRVDNNFCLRPMLAPNLYNYLRKLNRALPDPIKIFEIGPCYRKESDGKEHLEEFTMLNFCQMGSGCTRENLEAIIKDFLDYLGIDFEIVGDSCMVYGDTLDVMHGDLELSSAVVGPVPMDRDWGINKPWIGAGFGLERLLKVMHNFKNIKRASRSESYYNGISTNL

SEQ ID NO: 6 - protein, 416 aa, Methanococcoides burtonii
MEKQLLDVLVELNGVWLSRSGLLHGIRNFEITTKHIHIETDCGARFTVRNSRSSRSARSLRHNKYRKPCKRCRPADEQIDRFVKKTFKEKRQTVSVFSSPKKHVPKKPKVAVIKSFSISTPSPKEASVSNSIPTPSISVVKDEVKVPEVKYTPSQIERLKTLMSPDDKIPIQDELPEFKVLEKELIQRRRDDLKKMYEEDREDRLGKLERDITEFFVDRGFLEIKSPIMIPFEYIERMGIDKDDHLNKQIFRVDESMCLRPMLAPCLYNYLRKLDKVLPDPIRIFEIGPCYRKESDGSSHLEEFTMVNFCQMGSGCTRENMEALIDEFLEHLGIEYEIEADNCMVYGDTIDIMHGDLELSSAVVGPIPLDREWGVNKPWMGAGFGLERLLKVRHNYTNIRRASRSELYYNGINTNL

SEQ ID NO: 7 - protein, 279 aa, Desulfitobacterium hafniense
MSSFWTKVQYQRLKELNASGEQLEMGFSDALSRDRAFQGIEHQLMSQGKRHLEQLRTVKHRPALLELEEGLAKALHQQGFVQVVTPTIITKSALAKMTIGEDHPLFSQVFWLDGKKCLRPMLAPNLYTLWRELERLWDKPIRIFEIGTCYRKESQGAQHLNEFTMLNLTELGTPLEERHQRLEDMARWVLEAAGIREFELVTESSVVYGDTVDVMKGDLELASGAMGPHFLDEKWEIVDPWVGLGFGLERLLMIREGTQHVQSMARSLSYLDGVRLNIN

SEQ ID NO: 8 - protein, 312 aa, Desulfitobacterium hafniense
MDRIDHTDSKFVQAGETPVLPATFMFLTRRDPPLSSFWTKVQYQRLKELNASGEQLEMGFSDALSRDRAFQGIEHQLMSQGKRHLEQLRTVKHRPALLELEEGLAKALHQQGFVQVVTPTIITKSALAKMTIGEDHPLFSQVFWLDGKKCLRPMLAPNLYTLWRELERLWDKPIRIFEIGTCYRKESQGAQHLNEFTMLNLTELGTPLEERHQRLEDMARWVLEAAGIREFELVTESSVVYGDTVDVMKGDLELASGAMGPHFLDEKWEIVDPWVGLGFGLERLLMIREGTQHVQSMARSLSYLDGVRLNIN

SEQ ID NO: 9 - protein, 288 aa, Desulfitobacterium hafniense
MFLTRRDPPLSSFWTKVQYQRLKELNASGEQLEMGFSDALSRDRAFQGIEHQLMSQGKRHLEQLRTVKHRPALLELEEKLAKALHQQGFVQVVTPTIITKSALAKMTIGEDHPLFSQVFWLDGKKCLRPMLAPNLYTLWRELERLWDKPIRIFEIGTCYRKESQGAQHLNEFTMLNLTELGTPLEERHQRLEDMARWVLEAAGIREFELVTESSVVYGDTVDVMKGDLELASGAMGPHFLDEKWEIFDPWVGLGFGLERLLMIREGTQHVQSMARSLSYLDGVRLNIN

SEQ ID NO: 10 - protein, 277 aa, Desulfotomaculum acetoxidans
MSFLWTVSQQKRLSELNASEEEKNMSFSSTSDREAAYKRVEMRLINESKQRLNKLRHETRPAICALENRLAAALRGAGFVQVATPVILSKKLLGKMTITDEHALFSQVFWIEENKCLRPMLAPNLYYILKDLLRLWEKPVRIFEIGSCFRKESQGSNHLNEFTMLNLVEWGLPEEQRQKRISELAKLVMDETGIDEYHLEHAESVVYGETVDVMHRDIELGSGALGPHFLDGRWGVVGPWVGIGFGLERLLMVEQGGQNVRSMGKSLTYLDGVRLNI

SEQ ID NO: 11 - protein, 419 aa, Artificial Sequence
  Mb_AcKRS/1-419
MDKKPLDVLISATGLWMSRTGTLHKIKHHEVSRSKIYIEMACGDHLVVNNSRSCRTARAFRHHKYRKTCKRCRVSGEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERMGINNDTELSKQIFRVDKNLCLRPMVAPTIFNYARKLDRILPGPIKIFEVGPCYRKESDGKEHLEEFTMVNFFQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 12 - protein, 419 aa, Artificial Sequence
  Mb_PCKRS/1-419
MDKKPLDVLISATGLWMSRTGTLHKIKHHEVSRSKIYIEMACGDHLVVNNSRSCRTARAFRHHKYRKTCKRCRVSDEDINNFLTRSTESKNSVKVRVVSAPKVKKAMPKSVSRAPKPLENSVSAKASTNTSRSVPSPAKSTPNSSVPASAPAPSLTRSQLDRVEALLSPEDKISLNMAKPFRELEPELVTRRKNDFQRLYTNDREDYLGKLERDITKFFVDRGFLEIKSPILIPAEYVERFGINNDTELSKQIFRVDKNLCLRPMLSPTLCNYMRKLDRILPGPIKIFEVGPCYRKESDGKEHLEEFTMVNFCQMGSGCTRENLEALIKEFLDYLEIDFEIVGDSCMVYGDTLDIMHGDLELSSAVVGPVSLDREWGIDKPWIGAGFGLERLLKVMHGFKNIKRASRSESYYNGISTNL

SEQ ID NO: 13 - protein, 454 aa, Artificial Sequence
  Mm_AcKRS/1-454
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERMGIDNDTELSKQIFRVDKNFCLRPMVAPNIFNYARKLDRALPDPIKIFEIGPCYRKESDGKEHLEEFTMLNFFQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL

SEQ ID NO: 14 - protein, 454 aa, Artificial Sequence
  Mm_PCKRS/1-454
MDKKPLNTLISATGLWMSRTGTIHKIKHHEVSRSKIYIEMACGDHLVVNNSRSSRTARALRHHKYRKTCKRCRVSDEDLNKFLTKANEDQTSVKVKVVSAPTRTKKAMPKSVARAPKPLENTEAAQAQPSGSKFSPAIPVSTQESVSVPASVSTSISSISTGATASALVKGNTNPITSMSAPVQASAPALTKSQTDRLEVLLNPKDEISLNSGKPFRELESELLSRRKKDLQQIYAEERENYLGKLEREITRFFVDRGFLEIKSPILIPLEYIERFGIDNDTELSKQIFRVDKNFCLRPMLSPNLCNYMRKLDRALPDPIKIFEIGPCYRKESDGKEHLEEFTMLNFCQMGSGCTRENLESIITDFLNHLGIDFKIVGDSCMVYGDTLDVMHGDLELSSAVVGPIPLDREWGIDKPWIGAGFGLERLLKVKHDFKNIKRAARSESYYNGISTNL